SEQUENCE LISTING (120 sequences)

SEQ ID NO: 1 - RNA, 23 nt, Homo sapiens
uuaaugcuaaucgugauaggggu

SEQ ID NO: 2 - RNA, 22 nt, Homo sapiens
cuccuacauauuagcauuaaca

SEQ ID NO: 3 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (5)..(5): locked nucleic acid cytidine
  modified_base (6)..(6): locked nucleic acid guanosine
  modified_base (8)..(8): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 4 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (5)..(5): locked nucleic acid cytidine
  modified_base (6)..(6): locked nucleic acid guanosine
  modified_base (9)..(9): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 5 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (2)..(2): locked nucleic acid thymidine
  modified_base (6)..(6): locked nucleic acid guanosine
  modified_base (8)..(8): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 6 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (2)..(2): locked nucleic acid thymidine
  modified_base (6)..(6): locked nucleic acid guanosine
  modified_base (7)..(7): locked nucleic acid adenosine
  modified_base (10)..(10): locked nucleic acid adenosine
  modified_base (11)..(11): locked nucleic acid guanosine
  modified_base (12)..(12): locked nucleic acid cytidine
  modified_base (14)..(14): locked nucleic acid thymidine
  modified_base (16)..(16): locked nucleic acid adenosine
atcacgattagcatta

SEQ ID NO: 7 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (5)..(5): locked nucleic acid cytidine
  modified_base (6)..(6): locked nucleic acid guanosine
  modified_base (8)..(8): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (14)..(14): locked nucleic acid thymidine
  modified_base (16)..(16): locked nucleic acid adenosine
atcacgattagcatta

SEQ ID NO: 8 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (2)..(2): locked nucleic acid thymidine
  modified_base (5)..(5): locked nucleic acid cytidine
  modified_base (9)..(9): locked nucleic acid thymidine
  modified_base (10)..(10): locked nucleic acid adenosine
  modified_base (12)..(12): locked nucleic acid cytidine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 9 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (5)..(5): locked nucleic acid cytidine
  modified_base (7)..(7): locked nucleic acid adenosine
  modified_base (9)..(9): locked nucleic acid thymidine
  modified_base (10)..(10): locked nucleic acid adenosine
  modified_base (12)..(12): locked nucleic acid cytidine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 10 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (4)..(4): locked nucleic acid adenosine
  modified_base (7)..(7): locked nucleic acid adenosine
  modified_base (8)..(8): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 11 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (3)..(3): locked nucleic acid cytidine
  modified_base (6)..(6): locked nucleic acid guanosine
  modified_base (8)..(9): locked nucleic acid thymidine
  modified_base (12)..(12): locked nucleic acid cytidine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 12 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (2)..(2): locked nucleic acid thymidine
  modified_base (4)..(4): locked nucleic acid adenosine
  modified_base (5)..(5): locked nucleic acid cytidine
  modified_base (9)..(9): locked nucleic acid thymidine
  modified_base (10)..(10): locked nucleic acid adenosine
  modified_base (12)..(12): locked nucleic acid cytidine
  modified_base (13)..(13): locked nucleic acid adenosine
  modified_base (16)..(16): locked nucleic acid adenosine
atcacgattagcatta

SEQ ID NO: 13 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (3)..(3): locked nucleic acid cytidine
  modified_base (7)..(7): locked nucleic acid adenosine
  modified_base (9)..(9): locked nucleic acid thymidine
  modified_base (10)..(10): locked nucleic acid adenosine
  modified_base (12)..(12): locked nucleic acid cytidine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 14 - DNA, 16 bp, Artificial Sequence
  oligonucleotide inhibitor of miR-155
  misc_feature (1)..(16): joined through phosphorothioate bonds
  modified_base (1)..(1): locked nucleic acid adenosine
  modified_base (3)..(3): locked nucleic acid cytidine
  modified_base (5)..(5): locked nucleic acid cytidine
  modified_base (7)..(7): locked nucleic acid adenosine
  modified_base (9)..(9): locked nucleic acid thymidine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 15 - DNA, 15 bp, Artificial Sequence
  oligonucleotide inhibitor of miR-155
  misc_feature (1)..(15): joined through phosphorothioate bonds
  modified_base (1)..(1): locked nucleic acid thymidine
  modified_base (4)..(4): locked nucleic acid cytidine
  modified_base (7)..(7): locked nucleic acid thymidine
  modified_base (10)..(10): locked nucleic acid guanosine
  modified_base (12)..(12): locked nucleic acid adenosine
  modified_base (13)..(13): locked nucleic acid thymidine
  modified_base (15)..(15): locked nucleic acid adenosine
tcacgattagcatta

SEQ ID NO: 16 - DNA, 15 bp, Artificial Sequence
  oligonucleotide inhibitor of miR-155
  misc_feature (1)..(15): joined through phosphorothioate bonds
  modified_base (1)..(1): locked nucleic acid thymidine
  modified_base (3)..(3): locked nucleic acid adenosine
  modified_base (6)..(6): locked nucleic acid adenosine
  modified_base (7)..(7): locked nucleic acid thymidine
  modified_base (10)..(10): locked nucleic acid guanosine
  modified_base (12)..(12): locked nucleic acid adenosine
  modified_base (14)..(14): locked nucleic acid thymidine
  modified_base (15)..(15): locked nucleic acid adenosine
tcacgattagcatta

SEQ ID NO: 17 - DNA, 15 bp, Artificial Sequence
  oligonucleotide inhibitor of miR-155
  misc_feature (1)..(15): joined through phosphorothioate bonds
  modified_base (1)..(1): locked nucleic acid thymidine
  modified_base (5)..(5): locked nucleic acid guanosine
  modified_base (6)..(6): locked nucleic acid adenosine
  modified_base (7)..(7): locked nucleic acid thymidine
  modified_base (10)..(10): locked nucleic acid guanosine
  modified_base (12)..(12): locked nucleic acid adenosine
  modified_base (14)..(14): locked nucleic acid thymidine
  modified_base (15)..(15): locked nucleic acid adenosine
tcacgattagcatta

SEQ ID NO: 18 - DNA, 15 bp, Artificial Sequence
  oligonucleotide inhibitor of miR-155
  misc_feature (1)..(15): joined through phosphorothioate bonds
  modified_base (1)..(1): locked nucleic acid thymidine
  modified_base (2)..(2): locked nucleic acid cytidine
  modified_base (3)..(3): locked nucleic acid adenosine
  modified_base (5)..(5): locked nucleic acid guanosine
  modified_base (8)..(8): locked nucleic acid thymidine
  modified_base (9)..(9): locked nucleic acid adenosine
  modified_base (11)..(11): locked nucleic acid cytidine
  modified_base (14)..(14): locked nucleic acid thymidine
  modified_base (15)..(15): locked nucleic acid adenosine
tcacgattagcatta

SEQ ID NO: 19 - DNA, 15 bp, Artificial Sequence
  oligonucleotide inhibitor of miR-155
  misc_feature (1)..(15): joined through phosphorothioate bonds
  modified_base (1)..(1): locked nucleic acid thymidine
  modified_base (4)..(4): locked nucleic acid cytidine
  modified_base (8)..(8): locked nucleic acid thymidine
  modified_base (9)..(9): locked nucleic acid adenosine
  modified_base (10)..(10): locked nucleic acid guanosine
  modified_base (11)..(11): locked nucleic acid cytidine
  modified_base (12)..(12): locked nucleic acid adenosine
  modified_base (13)..(14): locked nucleic acid thymidine
  modified_base (15)..(15): locked nucleic acid adenosine
tcacgattagcatta

SEQ ID NO: 20 - DNA, 15 bp, Artificial Sequence
  oligonucleotide inhibitor of miR-155
  misc_feature (1)..(15): joined through phosphorothioate bonds
  modified_base (1)..(1): locked nucleic acid thymidine
  modified_base (3)..(3): locked nucleic acid adenosine
  modified_base (5)..(5): locked nucleic acid guanosine
  modified_base (6)..(6): locked nucleic acid adenosine
  modified_base (7)..(7): locked nucleic acid thymidine
  modified_base (10)..(10): locked nucleic acid guanosine
  modified_base (11)..(11): locked nucleic acid cytidine
  modified_base (12)..(12): locked nucleic acid adenosine
  modified_base (14)..(14): locked nucleic acid thymidine
  modified_base (15)..(15): locked nucleic acid adenosine
tcacgattagcatta

SEQ ID NO: 21 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (2)..(2): locked nucleic acid adenosine
  modified_base (3)..(4): locked nucleic acid thymidine
  modified_base (5)..(5): locked nucleic acid adenosine
  modified_base (6)..(6): locked nucleic acid guanosine
  modified_base (8)..(8): locked nucleic acid adenosine
  modified_base (9)..(9): locked nucleic acid thymidine
  modified_base (11)..(11): locked nucleic acid adenosine
gattagcatta

SEQ ID NO: 22 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (3)..(3): locked nucleic acid adenosine
  modified_base (4)..(5): locked nucleic acid thymidine
  modified_base (6)..(6): locked nucleic acid adenosine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 23 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (3)..(3): locked nucleic acid adenosine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 24 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (2)..(2): locked nucleic acid adenosine
  modified_base (4)..(4): locked nucleic acid guanosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 25 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 26 - DNA, 15 bp, Artificial Sequence
  oligonucleotide inhibitor of miR-155
  misc_feature (1)..(15): joined through phosphorothioate bonds
  modified_base (1)..(1): locked nucleic acid thymidine
  modified_base (3)..(3): locked nucleic acid adenosine
  modified_base (4)..(4): 5-methyl deoxycytidine
  modified_base (5)..(5): locked nucleic acid guanosine
  modified_base (6)..(6): locked nucleic acid adenosine
  modified_base (7)..(7): locked nucleic acid thymidine
  modified_base (10)..(10): locked nucleic acid guanosine
  modified_base (11)..(11): locked nucleic acid cytidine
  modified_base (12)..(12): locked nucleic acid adenosine
  modified_base (14)..(14): locked nucleic acid thymidine
  modified_base (15)..(15): locked nucleic acid adenosine
tcacgattagcatta

SEQ ID NO: 27 - DNA, 8 bp, Artificial Sequence
  oligonucleotide inhibitor of miR-155
  misc_feature (1)..(8): joined through phosphorothioate bonds
  modified_base (1)..(1): locked nucleic acid thymidine
  modified_base (2)..(2): locked nucleic acid adenosine
  modified_base (3)..(3): locked nucleic acid guanosine
  modified_base (4)..(4): locked nucleic acid cytidine
  modified_base (5)..(5): locked nucleic acid adenosine
  modified_base (6)..(7): locked nucleic acid thymidine
  modified_base (8)..(8): locked nucleic acid adenosine
tagcatta

SEQ ID NO: 28 - skipped

SEQ ID NO: 29 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (5)..(5): locked nucleic acid adenosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 30 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (5)..(5): locked nucleic acid adenosine
  modified_base (7)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 31 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (8)..(8): locked nucleic acid adenosine
  modified_base (9)..(9): locked nucleic acid guanosine
  modified_base (11)..(11): locked nucleic acid adenosine
  modified_base (13)..(13): locked nucleic acid thymidine
  modified_base (14)..(14): locked nucleic acid adenosine
cacgattagcatta

SEQ ID NO: 32 - DNA, 14 bp, Artificial Sequence
  oligonucleotide inhibitor of miR-155
  misc_feature (1)..(14): joined through phosphorothioate bonds
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 33 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (2)..(2): locked nucleic acid adenosine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 34 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 35 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (4)..(4): locked nucleic acid guanosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 36 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (5)..(5): locked nucleic acid adenosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 37 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (7)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 38 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(6): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 39 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (8)..(8): locked nucleic acid adenosine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 40 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 41 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (10)..(10): locked nucleic acid cytidine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 42 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (12)..(13): locked nucleic acid thymidine
  modified_base (14)..(14): locked nucleic acid adenosine
cacgattagcatta

SEQ ID NO: 43 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (13)..(13): locked nucleic acid thymidine
  modified_base (14)..(14): locked nucleic acid adenosine
cacgattagcatta

SEQ ID NO: 44 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(12): locked nucleic acid thymidine
  modified_base (14)..(14): locked nucleic acid adenosine
cacgattagcatta

SEQ ID NO: 45 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
cacgattagcatta

SEQ ID NO: 46 - DNA, 14 bp, Artificial Sequence
  oligonucleotide inhibitor of miR-155
  misc_feature (1)..(14): joined through phosphorothioate bonds
  modified_base (2)..(2): locked nucleic acid adenosine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 47 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (2)..(2): locked nucleic acid adenosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 48 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (4)..(4): locked nucleic acid guanosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 49 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (5)..(5): locked nucleic acid adenosine
  modified_base (7)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 50 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(6): locked nucleic acid thymidine
  modified_base (8)..(8): locked nucleic acid adenosine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 51 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (8)..(8): locked nucleic acid adenosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 52 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (10)..(10): locked nucleic acid cytidine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 53 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (10)..(10): locked nucleic acid cytidine
  modified_base (12)..(13): locked nucleic acid thymidine
  modified_base (14)..(14): locked nucleic acid adenosine
cacgattagcatta

SEQ ID NO: 54 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 55 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 56 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 57 - DNA, 13 bp, Artificial Sequence
  oligonucleotide inhibitor of miR-155
  misc_feature (1)..(13): joined through phosphorothioate bonds
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 58 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 59 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (3)..(3): locked nucleic acid guanosine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 60 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (4)..(4): locked nucleic acid adenosine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 61 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (6)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 62 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(5): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 63 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (7)..(7): locked nucleic acid adenosine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 64 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 65 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (9)..(9): locked nucleic acid cytidine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 66 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (11)..(12): locked nucleic acid thymidine
  modified_base (13)..(13): locked nucleic acid adenosine
acgattagcatta

SEQ ID NO: 67 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (12)..(12): locked nucleic acid thymidine
  modified_base (13)..(13): locked nucleic acid adenosine
acgattagcatta

SEQ ID NO: 68 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(11): locked nucleic acid thymidine
  modified_base (13)..(13): locked nucleic acid adenosine
acgattagcatta

SEQ ID NO: 69 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
acgattagcatta

SEQ ID NO: 70 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (3)..(3): locked nucleic acid guanosine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 71 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (4)..(4): locked nucleic acid adenosine
  modified_base (6)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 72 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 73 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(5): locked nucleic acid thymidine
  modified_base (7)..(7): locked nucleic acid adenosine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 74 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (7)..(7): locked nucleic acid adenosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 75 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (9)..(9): locked nucleic acid cytidine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 76 - DNA, 13 bp, Artificial Sequence
  oligonucleotide inhibitor of miR-155
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (9)..(9): locked nucleic acid cytidine
  modified_base (11)..(12): locked nucleic acid thymidine
  modified_base (13)..(13): locked nucleic acid adenosine
acgattagcatta

SEQ ID NO: 77 - DNA, 12 bp, Artificial Sequence
  oligonucleotide inhibitor of miR-155
  misc_feature (1)..(12): joined through phosphorothioate bonds
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 78 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (2)..(2): locked nucleic acid guanosine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 79 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (3)..(3): locked nucleic acid adenosine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 80 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (5)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 81 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(4): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 82 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (6)..(6): locked nucleic acid adenosine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 83 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 84 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (8)..(8): locked nucleic acid cytidine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 85 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (10)..(11): locked nucleic acid thymidine
  modified_base (12)..(12): locked nucleic acid adenosine
cgattagcatta

SEQ ID NO: 86 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (9)..(9): locked nucleic acid adenosine
  modified_base (11)..(11): locked nucleic acid thymidine
  modified_base (12)..(12): locked nucleic acid adenosine
cgattagcatta

SEQ ID NO: 87 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (9)..(9): locked nucleic acid adenosine
  modified_base (10)..(10): locked nucleic acid thymidine
  modified_base (12)..(12): locked nucleic acid adenosine
cgattagcatta

SEQ ID NO: 88 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
cgattagcatta

SEQ ID NO: 89 - DNA, 12 bp, Artificial Sequence
  oligonucleotide inhibitor of miR-155
  misc_feature (1)..(12): joined through phosphorothioate bonds
  modified_base (2)..(2): locked nucleic acid guanosine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 90 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (3)..(3): locked nucleic acid adenosine
  modified_base (5)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 91 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(4): locked nucleic acid thymidine
  modified_base (6)..(6): locked nucleic acid adenosine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 92 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (6)..(6): locked nucleic acid adenosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 93 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (8)..(8): locked nucleic acid cytidine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 94 - DNA, 12 bp, Artificial Sequence
  oligonucleotide inhibitor of miR-155
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (8)..(8): locked nucleic acid cytidine
  modified_base (10)..(11): locked nucleic acid thymidine
  modified_base (12)..(12): locked nucleic acid adenosine
cgattagcatta

SEQ ID NO: 95 - DNA, 11 bp, Artificial Sequence
  oligonucleotide inhibitor of miR-155
  misc_feature (1)..(11): joined through phosphorothioate bonds
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 96 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (2)..(2): locked nucleic acid adenosine
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 97 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (4)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 98 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(3): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 99 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (5)..(5): locked nucleic acid adenosine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 100 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 101 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (7)..(7): locked nucleic acid cytidine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 102 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (9)..(10): locked nucleic acid thymidine
  modified_base (11)..(11): locked nucleic acid adenosine
gattagcatta

SEQ ID NO: 103 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (8)..(8): locked nucleic acid adenosine
  modified_base (10)..(10): locked nucleic acid thymidine
  modified_base (11)..(11): locked nucleic acid adenosine
gattagcatta

SEQ ID NO: 104 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (8)..(8): locked nucleic acid adenosine
  modified_base (9)..(9): locked nucleic acid thymidine
  modified_base (11)..(11): locked nucleic acid adenosine
gattagcatta

SEQ ID NO: 105 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
gattagcatta

SEQ ID NO: 106 - DNA, 11 bp, Artificial Sequence
  oligonucleotide inhibitor of miR-155
  misc_feature (1)..(11): joined through phosphorothioate bonds
  modified_base (2)..(2): locked nucleic acid adenosine
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 107 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (2)..(2): locked nucleic acid adenosine
  modified_base (4)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 108 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(3): locked nucleic acid thymidine
  modified_base (5)..(5): locked nucleic acid adenosine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 109 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (5)..(5): locked nucleic acid adenosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 110 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (7)..(7): locked nucleic acid cytidine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 111 - DNA, 11 bp, Artificial Sequence
  oligonucleotide inhibitor of miR-155
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (7)..(7): locked nucleic acid cytidine
  modified_base (9)..(10): locked nucleic acid thymidine
  modified_base (11)..(11): locked nucleic acid adenosine
gattagcatta

SEQ ID NO: 112 - DNA, 14 bp, Artificial Sequence
  oligonucleotide inhibitor of miR-155
  misc_feature (1)..(14): joined through phosphorothioate bonds
  modified_base (1)..(1): ethylene-bridged cytidine
  modified_base (3)..(3): ethylene-bridged cytidine
  modified_base (6)..(7): ethylene-bridged thymidine
  modified_base (9)..(9): ethylene-bridged guanosine
  modified_base (11)..(11): ethylene-bridged adenosine
  modified_base (12)..(13): ethylene-bridged thymidine
  modified_base (14)..(14): ethylene-bridged adenosine
cacgattagcatta

SEQ ID NO: 113 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): ethylene-bridged adenosine
  modified_base (12)..(13): locked nucleic acid thymidine
  modified_base (14)..(14): ethylene-bridged adenosine
cacgattagcatta

SEQ ID NO: 114 - DNA, 14 bp, Artificial Sequence
  oligonucleotide inhibitor of miR-155
  misc_feature (1)..(14): joined through phosphorothioate bonds
  modified_base (1)..(1): ethylene-bridged cytidine
  modified_base (3)..(3): ethylene-bridged cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 115 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): ethylene-bridged guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 116 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): ethylene-bridged thymidine
  modified_base (9)..(9): locked nucleic acid guanosine
  modified_base (11)..(11): locked nucleic acid adenosine
  modified_base (12)..(13): ethylene-bridged thymidine
  modified_base (14)..(14): locked nucleic acid adenosine
cacgattagcatta

SEQ ID NO: 117 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 118 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(12): locked nucleic acid thymidine
  modified_base (13)..(13): ethylene-bridged thymidine
  modified_base (14)..(14): locked nucleic acid adenosine
cacgattagcatta

SEQ ID NO: 119 - DNA, 14 bp, Artificial Sequence
  oligonucleotide inhibitor of miR-155
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): amino-2′-C-bridged bicyclic adenosine
  modified_base (12)..(13): locked nucleic acid thymidine
  modified_base (14)..(14): amino-2′-C-bridged bicyclic adenosine
cacgattagcatta

SEQ ID NO: 120 - DNA, 14 bp, Artificial Sequence
  oligonucleotide inhibitor of miR-155
  misc_feature (1)..(14): joined through phosphorothioate bonds
  modified_base (1)..(1): amino-2′-C-bridged bicyclic cytidine
  modified_base (3)..(3): amino-2′-C-bridged bicyclic cytidine
  modified_base (6)..(7): amino-2′-C-bridged bicyclic thymidine
  modified_base (9)..(9): amino-2′-C-bridged bicyclic guanosine
  modified_base (11)..(11): amino-2′-C-bridged bicyclic adenosine
  modified_base (12)..(13): amino-2′-C-bridged bicyclic thymidine
  modified_base (14)..(14): amino-2′-C-bridged bicyclic adenosine
cacgattagcatta

(19) United States
(12) Patent Application Publication
(10) Pub. No.: US 2020/0276220 A1
(43) Pub. Date: Sep. 3, 2020

(54) METHODS FOR TREATING CUTANEOUS T-CELL LYMPHOMA (CTCL) WITH MIR-155 INHIBITORS

(71) Applicant: MIRAGEN THERAPEUTICS, INC., Boulder, CO (US)

(72) Inventors: Aimee L. JACKSON, Boulder, CO (US); William S. MARSHALL, Boulder, CO (US)

(21) Appl. No.: 16/315,344

(22) PCT Filed: Jul. 7, 2017
(86) PCT No.: PCT/US2017/041180

§ 371 Date: Jan. 4, 2019

Related U.S. Application Data
(60) Provisional application No. 62/429,687, filed on Dec. 2, 2016.
(60) Provisional application No. 62/359,499, filed on Jul. 7, 2016.

(51) Int. Cl. A61K 31/711; A61K 9/00; A61K 45/06; A61P 35/00; A61K 31/192; A61K 31/167; A61K 38/15; A61K 31/4045; A61K 31/506; A61K 31/18; A61K 31/343; A61K 31/4406

(52) U.S. Cl. CPC A61K 31/711; A61K 9/0019; A61K 45/06; A61P 35/00; A61K 31/192; A61K 31/4406; A61K 38/15; A61K 31/4045; A61K 31/506; A61K 31/18; A61K 31/343; A61K 31/167

(57) ABSTRACT

The present invention provides compositions and methods for treating cutaneous T-cell lymphoma (CTCL) with intralesional administration of one or more miR-155 inhibitors. In certain embodiments, the intralesional administration of one or more oligonucleotide inhibitors of miR-155 reduces the redness, thickness, height, scaling, and/or surface area of one or more untreated lesions on the skin of said subject.

### CROSS-REFERENCE

This application is a 371 national stage application of PCT Application No. PCT/US2017/041180, filed Jul. 7, 2017, which claims the priority benefit of U.S. Provisional Patent Application Ser. No. 62/359,499, filed on Jul. 7, 2016, and U.S. Provisional Patent Application Ser. No. 62/429,687, filed on Dec. 2, 2016, all of which are hereby incorporated by reference in their entirety.

### REFERENCE TO SEQUENCE LISTING

The contents of the text file submitted electronically herewith are incorporated herein by reference in their entirety: A computer readable format copy of the Sequence Listing (filename: MIRG_054_02US_SeqList_ST25.txt, date recorded: Aug. 14, 2019, file size ˜106 kilobytes).

### BACKGROUND OF THE INVENTION

Cutaneous lymphomas of T-cell or B-cell origin comprise approximately 3.9% of all non-Hodgkin's lymphomas. Of these, approximately 53% are of T-cell origin (cutaneous T-cell lymphoma or CTCL). The prevalence of the disease in the United States is estimated at 30,000 cases (Cutaneous Lymphoma Foundation, 2014, “Cutaneous Lymphoma Fast Facts.”), although this estimate is acknowledged to be low due to the difficulty of diagnosis in the early stages of the disease. The annual age-adjusted incidence of CTCL in the United States is estimated at 6.4-9.6 cases per million people (Jawed, et al., 2014, J Am Acad Dermatol 70(2): 205 e201-16).

Chronic inflammation is a critical hallmark of CTCL, a clinically heterogeneous group of lymphoproliferative malignancies that are characterized by localization of neoplastic T lymphocytes to the skin. Mycosis fungoides (MF) is the most prevalent sub-type of CTCL, accounting for 50-70% of all primary cutaneous lymphomas. In this sub-type, CTCL presents in the skin with no evidence of extracutaneous disease at the time of diagnosis. The second most prevalent sub-type is Sézary Syndrome (SS), comprising 15% of CTCL cases. MF is characterized by proliferation of atypical small- to medium-sized T lymphocytes with cerebriform nuclei that form patches, plaques, or nodular tumors in the epidermis. MF typically affects older adults (median age of diagnosis: 55-60) and has an indolent clinical course where patches and plaques precede or are concurrent with the formation of tumors. In some late tumor-stage cases, lymph node and visceral organ involvement are observed. During tumor-stage MF, the dermal infiltrates become more diffuse and the epidermotropism of the atypical T-cells may be lost. In contrast, SS is a more aggressive, leukemic form of CTCL, characterized by widespread redness and scaling of the skin (erythroderma), enlarged lymph nodes, and malignant cells in the peripheral circulation (Yamashita, et al., 2012, An Bras Dermatol 87(6): 817-28, Jewad et al., 2014, supra).

CTCL is characterized by aberrant expression and function of transcription factors and regulators of signal transduction. It has been hypothesized that dysfunctional regulation of signal molecules and cytokines plays a key role in the malignant transformation and pathogenesis of CTCL (Girardi et al., 2004, N Engl J Med 350(19): 1978-88, Zhang et al., 2006, Blood 108(3): 1058-64, van Doom et al., 2009, Blood 113(1): 127-36, and Kadin et al., 2010, Nat Rev Clin Oncol 7(8): 430-2). Significant differences in the gene expression profiles of MF and SS cells have been observed, consistent with a distinct pathogenesis for these variants of CTCL (van Doom et al., 2009, supra, Campbell et al., 2010, Blood 116(5): 767-71).

Recently, microRNAs have been reported to be differentially expressed and potentially involved in the pathogenesis of CTCL. The selection of inhibitors of microRNAs, routes of administration, and dosing paradigms for treatment of CTCL remains a significant challenge. Accordingly, the present invention provides compositions and methods for treating CTCL with microRNA inhibitors.

Patents, patent applications, patent application publications, journal articles and protocols referenced herein are incorporated by reference in their entireties, for all purposes.

### BRIEF SUMMARY OF THE INVENTION

The present invention provides methods for treating CTCL with intralesional administration of one or more miR-155 inhibitors.

In one embodiment, the present invention provides a method of treating cutaneous T-cell lymphoma (CTCL) in a subject in need thereof, wherein the method comprises administering to the subject an oligonucleotide inhibitor of miR-155, wherein the inhibitor is administered intralesionally, i.e., intratumorally. In an exemplary embodiment, the inhibitor is administered via intralesional injection. In certain embodiments, the CTCL is the mycosis fungoides (MF) form of CTCL.

In some embodiments, the intralesional administration of one or more oligonucleotide inhibitors of miR-155 provides therapeutic benefits to untreated lesions on the skin of the subject. In certain embodiments, the intralesional administration of one or more oligonucleotide inhibitors of miR-155 reduces the redness, thickness, height, scaling, and/or surface area of one or more untreated lesions on the skin of said subject.

In some embodiments, the present invention provides a method of treating cutaneous T-cell lymphoma (CTCL) in a subject in need thereof, wherein the method comprises intralesionally administering to the subject an oligonucleotide inhibitor of miR-155, and further comprises administering one or more therapeutic agents subcutaneously and/or intravenously. In some embodiments, the second therapeutic agent is an oligonucleotide inhibitor of miR-155. In one embodiment, the oligonucleotide inhibitor of miR-155 that is administered intralesionally is the same oligonucleotide inhibitor of miR-155 that is administered subcutaneously and/or intravenously. In an alternative embodiment, the oligonucleotide inhibitor of miR-155 that is administered intralesionally is different than the oligonucleotide inhibitor of miR-155 that is administered subcutaneously and/or intravenously. In other embodiments, the second therapeutic agent is a retinoid or a histone deacetylase (HDAC) inhibitor.

In some embodiments, the oligonucleotide inhibitor of miR-155 is formulated with a pharmaceutically acceptable carrier or excipient. In one embodiment, the oligonucleotide inhibitor of miR-155 is formulated for administration at a concentration of about 10 mg/mL to a concentration of about 500 mg/mL. In another embodiment, the oligonucleotide inhibitor of miR-155 is formulated for administration at a concentration of about 20 mg/mL to a concentration of about 200 mg/mL. In an exemplary embodiment, the oligonucleotide inhibitor of miR-155 is formulated for administration at a concentration of about 75 mg/mL. In another exemplary embodiment, the oligonucleotide inhibitor of miR-155 is formulated for administration at a concentration of about 150 mg/mL.

In some embodiments, the oligonucleotide inhibitor of miR-155 comprises a sequence of about 8 nucleotides to about 22 nucleotides that is at least partially complementary to a mature sequence of miR-155-5p, e.g. at least about 75%, 80%, 85%, 90%, 95%, 96%, 97%, 98%, or 99% complementary to a mature sequence of miR-155-5p. In one embodiment, the oligonucleotide inhibitor comprises a sequence that is 100% or fully complementary to a mature sequence of miR-155-5p. In some embodiments, the oligonucleotide inhibitor of miR-155 comprises one or more modified nucleotides. The modified nucleotides that may be present in the oligonucleotide inhibitors of the present invention include, but are not limited to, locked nucleotides, ethylene-bridged nucleotides, 2′-C-bridged bicyclic nucleotides, 2′-substituted nucleotides, and other sugar and/or base modifications described herein. In some embodiments, all modified nucleotides present in the oligonucleotide inhibitors of the present invention are locked nucleotides. In some other embodiments, modified nucleotides present in the oligonucleotide inhibitors are a combination of locked nucleotides and other modifications such as ethylene-bridged nucleotides, 2′-C-bridged bicyclic nucleotides, and 2′-substituted nucleotides, and other sugar and/or base modifications described herein.

In one embodiment, the oligonucleotide inhibitor of miR-155 comprises a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of the oligonucleotide inhibitor are locked nucleotides and at least the second nucleotide from the 5′ end of the oligonucleotide inhibitor is a deoxyribonucleic acid (DNA) nucleotide. In some of these embodiments, the fourth nucleotide from the 3′ end of the oligonucleotide inhibitor is also a locked nucleotide. In some of these embodiments, the first nucleotide from the 5′ end of the oligonucleotide inhibitor is a locked nucleotide.

In another embodiment, the oligonucleotide inhibitor of miR-155 comprises a sequence of 11 to 14 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are modified nucleotides and at least the second nucleotide from the 5′ end of the oligonucleotide inhibitor is a deoxyribonucleic acid (DNA) nucleotide. In these embodiments, the oligonucleotide inhibitor may contain at least 5, 6, 7, 8, 9, or 10 modified nucleotides. In some of these embodiments, the oligonucleotide inhibitor contains 7, 8, 9, or 10 modified nucleotides. In some of these embodiments, 7, 8, 9, or 10 modified nucleotides present in the oligonucleotide inhibitor are all locked nucleotides. In yet some other embodiments, 7, 8, 9, or 10 modified nucleotides present in the oligonucleotide inhibitor are a combination of locked nucleotides and other modifications such as ethylene-bridged nucleotides, 2′-C-bridged bicyclic nucleotides, and sugar modifications such as 2′-substituted nucleotides. In some of these embodiments, the second DNA nucleotide from the 5′ end of the oligonucleotide inhibitor could be an unmodified DNA nucleotide. In some of these embodiments, the first three modified nucleotides from the 3′ end of the oligonucleotide inhibitor are locked nucleotides.

In yet another embodiment, the oligonucleotide inhibitor of miR-155 comprises a sequence of 11 to 14 nucleotides, wherein the oligonucleotide inhibitor is fully complementary to a mature sequence of miR-155 and has a full phosphorothioate backbone; and wherein at least 7 nucleotides of said oligonucleotide inhibitor are modified nucleotides and at least the second nucleotide from the 5′ end of the oligonucleotide inhibitor is a deoxyribonucleic acid (DNA) nucleotide. In these embodiments, the oligonucleotide inhibitor may contain at least 7, 8, 9, or 10 modified nucleotides. In some of these embodiments, 7, 8, 9, or 10 modified nucleotides present in the oligonucleotide inhibitor are all locked nucleotides. In yet some other embodiments, 7, 8, 9, or 10 modified nucleotides present in the oligonucleotide inhibitor are a combination of locked nucleotides and other modifications such as ethylene-bridged nucleotides, 2′-C-bridged bicyclic nucleotides, and sugar modifications such as 2′-substituted nucleotides. In some of these embodiments, the first three nucleotides from the 3′ end of the oligonucleotide inhibitor are modified nucleotides. In some of these embodiments, the first three modified nucleotides from the 3′ end of the oligonucleotide inhibitor are locked nucleotides. In some of these embodiments, the second or the third nucleotide from the 3′ end of the oligonucleotide inhibitor is a DNA nucleotide. In some of these embodiments, the second DNA nucleotide from the 5′ end of the oligonucleotide inhibitor could be an unmodified DNA nucleotide.

In yet another embodiment, the oligonucleotide inhibitor of miR-155 comprises a sequence of 11 to 14 nucleotides, wherein the oligonucleotide inhibitor is fully complementary to a mature sequence of miR-155 and has a full phosphorothioate backbone; and wherein at least the first three nucleotides from 3′ end of said oligonucleotide inhibitor are modified nucleotides and at least the fourth and fifth nucleotides from the 5′ end of the oligonucleotide inhibitor are deoxyribonucleic acid (DNA) nucleotides. In these embodiments, the oligonucleotide inhibitor may contain at least 7, 8, 9, or 10 modified nucleotides. In some of these embodiments, 7, 8, 9, or 10 modified nucleotides present in the oligonucleotide inhibitor are all locked nucleotides. In some of these embodiments, the first three modified nucleotides from the 3′ end of the oligonucleotide inhibitor are locked nucleotides. In some of these embodiments, the fourth and/or the fifth DNA nucleotide from the 5′ end of the oligonucleotide inhibitor could be an unmodified DNA nucleotide.

In one embodiment, the oligonucleotide inhibitor of miR-155 has a length of 12 to 14 nucleotides. In some embodiments, the oligonucleotide inhibitor contains at least 5, 6, 7, 8, 9 or 10 locked nucleotides. In some other embodiments, the oligonucleotide inhibitor contains at least 1, 2, 3, 4, 5, or more DNA nucleotides. In certain embodiments, at least the second nucleotide from the 5′ end of the oligonucleotide inhibitor is a DNA nucleotide. In certain additional embodiments, at least the second and fourth nucleotides from the 5′ end of the oligonucleotide inhibitor are DNA nucleotides. In further embodiments, at least the sixth and/or the eighth nucleotide from the 5′ end of the oligonucleotide inhibitor is a DNA nucleotide. In yet further embodiments, the oligonucleotide inhibitor comprises DNA nucleotides at the second, sixth, and the eighth position from the 5′ end.

In some embodiments, the oligonucleotide inhibitor of miR-155 has a sequence selected from SEQ ID NOs: 3-27 and 29-120. In an exemplary embodiment, the oligonucleotide inhibitor of miR-155 has a sequence of SEQ ID NO: 25. In another exemplary embodiment, the oligonucleotide inhibitor of miR-155 has a sequence of SEQ ID NO: 22 or 23. In yet another exemplary embodiment, the oligonucleotide inhibitor of miR-155 has a sequence selected from SEQ ID NO: 33, 39, 43, 44, 47, 58, 84, 99, 111, 115, and 120.

In one embodiment, the invention provides methods for reducing or inhibiting the proliferation of malignant T cells (e.g., CTCL cells), comprising intralesionally administering an oligonucleotide inhibitor of miR-155. The activity or function of miR-155 is reduced in malignant T cells (e.g., CTCL cells) following administration of the oligonucleotide inhibitor.

### BRIEF DESCRIPTION OF THE DRAWINGS

FIG. 1 provides the study design of the clinical trial.

FIG. 2 shows the efficacy of intratumoral injection of the miR-155 inhibitor.

FIG. 3 shows the efficacy of intratumoral injection of the miR-155 inhibitor.

FIG. 4 shows a photographic example of a clinical response for a patient in the clinical trial.

FIG. 5 shows the histological findings and changes in pruritus after 8 or 15 days of miR-155 treatment.

FIG. 6 shows miR-155 copy number in baseline lesion biopsies.

FIG. 7 shows the gene expression changes common to mycosis fungoides lesion biopsies with intratumoral injection of the miR-155 inhibitor.

FIG. 8 shows that miR-155 inhibitor treatment inactivates certain pathways.

### DETAILED DESCRIPTION

The present inventors have now discovered that administration of a miR-155 inhibitor intratumorally (hereinafter referred to interchangeably as intralesional) promotes regression of the injected lesion as well as non-injected lesions in multiple human patients with CTCL. Direct intratumoral administration of drug has not been shown previously to have beneficial effects on distal lesions in CTCL patients. (Rook et al., 1999, Blood 94(3): 902-8) Intratumoral administration of a miR-155 inhibitor provides a broad clinical response on lesions beyond the injected lesion. Accordingly, the present invention provides compositions and methods for treating CTCL with intratumoral administration of one or more miR-155 inhibitors.

Specifically, the present invention provides methods for treating CTCL with intralesional administration of one or more oligonucleotide inhibitors that inhibit the activity or function of miR-155 in CTCL cancer cells. The methods for treating CTCL comprise intralesionally administering to a subject an oligonucleotide inhibitor of miR-155 that inhibits the activity or function of miR-155 in CTCL cancer cells.

In humans, miR-155 is encoded by the MIR155 host gene or MIR155HG and is located on human chromosome 21. Since both arms of pre-miR-155 can give rise to mature miRNAs, processing products of pre-miR-155 are designated as miR-155-5p (from the 5′ arm) and miR-155-3p (from the 3′ arm). The mature sequences for human miR-155-5p and miR-155-3p are given below:

Human mature miR-155-5p
(SEQ ID NO: 1)
5′-UUAAUGCUAAUCGUGAUAGGGGU-3′
Human mature miR-155-3p
(SEQ ID NO: 2)
5′-CUCCUACAUAUUAGCAUUAACA-3′

miR-155-5p is expressed in hematopoietic cells including B-cells, T-cells, monocytes and granulocytes (Landgraf et al. 2007). miR-155-5p plays a role in mediating inflammatory and immune responses.

In the context of the present invention, the term “oligonucleotide inhibitor”, “antimiR” (e.g., antimiR-155), “antagonist”, “antisense oligonucleotide or ASO”, “oligomer”, “anti-microRNA oligonucleotide or AMO”, or “mixmer” is used broadly and encompasses an oligomer comprising ribonucleotides, deoxyribonucleotides, modified ribonucleotides, modified deoxyribonucleotides or a combination thereof, that inhibits the activity or function of the target microRNA (miRNA) by fully or partially hybridizing to the miRNA thereby repressing the function or activity of the target miRNA.

The term “miR-155” as used herein includes pri-miR-155, pre-miR-155, miR-155-5p, and hsa-miR-155-5p.

In various embodiments described herein, the oligonucleotide inhibitor of miR-155 is administered intralesionally, i.e., intratumorally. In an exemplary embodiment, the inhibitor is administered via intralesional injection. As used herein, and in the context of the treatment of CTCL, the terms “intralesional” or “intralesionally” can be used interchangeably with “intratumoral” or “intratumorally” and refer to administering the oligonucleotide inhibitor of miR-155 directly into a CTCL skin lesion. In certain embodiments, the CTCL is the mycosis fungoides (MF) form of CTCL.

In some embodiments, the intralesional administration of one or more oligonucleotide inhibitors of miR-155 provides therapeutic benefits to both treated lesions (i.e., injected lesions) and untreated lesions (i.e., non-injected lesions) on the skin of the subject. In some embodiments, the treatment methods described herein reduce one or more of the following: (i) the coloration, for example, redness, of a lesion, (ii) the thickness of a lesion, (iii) the height of a lesion, (iv) the amount of scaling at the site of a lesion, and/or (v) the surface area of a lesion. In one embodiment, the reduction in one or more of the aforementioned characteristics occurs at the site of the treated lesion. In another embodiment, the reduction in one or more of the aforementioned characteristics occurs at the site(s) of one or more untreated lesions. In yet another embodiment, the reduction in one or more of the aforementioned characteristics occurs at the site of the treated lesion as well as at the site(s) of one or more untreated lesions.

The sequence of an oligonucleotide inhibitor of miR-155 for use in the methods of the present invention is preferably sufficiently complementary to a mature sequence of miR-155-5p to hybridize to miR-155-5p under physiological conditions and inhibit the activity or function of miR-155-5p in the cells of a subject. For instance, in some embodiments, oligonucleotide inhibitors comprise a sequence that is at least partially complementary to a mature sequence of miR-155-5p, e.g. at least about 75%, 80%, 85%, 90%, 95%, 96%, 97%, 98%, or 99% complementary to a mature sequence of miR-155-5p. In some embodiments, the oligonucleotide inhibitor can be substantially complementary to a mature sequence of miR-155-5p, that is at least about 90%, 95%, 96%, 97%, 98%, or 99% complementary to a mature sequence of miR-155-5p. In one embodiment, the oligonucleotide inhibitor comprises a sequence that is 100% or fully complementary to a mature sequence of miR-155-5p. It is understood that the sequence of the oligonucleotide inhibitor is considered to be complementary to miR-155 even if the oligonucleotide sequence includes a modified nucleotide instead of a naturally-occurring nucleotide. For example, if a mature sequence of miR-155 comprises a guanosine nucleotide at a specific position, the oligonucleotide inhibitor may comprise a modified cytidine nucleotide, such as a locked cytidine nucleotide or 2′-fluoro-cytidine, at the corresponding position.

In some embodiments, the oligonucleotide inhibitor of miR-155 comprises a sequence of about 8 nucleotides to about 22 nucleotides that is at least partially complementary to a mature sequence of miR-155-5p, e.g. at least about 75%, 80%, 85%, 90%, 95%, 96%, 97%, 98%, or 99% complementary to a mature sequence of miR-155-5p. The term “about” as used herein encompasses variations of +/−10% and more preferably +/−5%, as such variations are appropriate for practicing the present invention.

In various embodiments, the oligonucleotide inhibitor targeting miR-155 is 8, 9, 10, 11, 12, 13, 14, 15, 16, 17, 18, 19, 20, 21, or 22 nucleotides in length. In one embodiment, the oligonucleotide inhibitor comprises a sequence that is 100% or fully complementary to a mature sequence of miR-155-5p. In some embodiments, the oligonucleotide inhibitor of miR-155 comprises one or more modified nucleotides. The modified nucleotides that may be present in the oligonucleotide inhibitors of the present invention include, but are not limited to, locked nucleotides, ethylene-bridged nucleotides, 2′-C-bridged bicyclic nucleotides, 2′-substituted nucleotides, and other sugar and/or base modifications described herein. In some embodiments, all modified nucleotides present in the oligonucleotide inhibitors of the present invention are locked nucleotides. In some other embodiments, modified nucleotides present in the oligonucleotide inhibitors are a combination of locked nucleotides and other modifications such as ethylene-bridged nucleotides, 2′-C-bridged bicyclic nucleotides, and 2′-substituted nucleotides, and other sugar and/or base modifications described herein.

In certain embodiments, the oligonucleotide inhibitor of miR-155 for use in the methods of the present invention has a length of 11 to 16 nucleotides. In some other embodiments, the oligonucleotide inhibitor of miR-155 for use in the methods of the present invention has a length of 11 to 14 nucleotides. In various embodiments, the oligonucleotide inhibitor targeting miR-155 is 11, 12, 13, 14, 15, or 16 nucleotides in length. In one embodiment, the oligonucleotide inhibitor of miR-155 has a length of 12 nucleotides. In another embodiment, the oligonucleotide inhibitor of miR-155 has a length of 14 nucleotides.

In some embodiments, the entire sequence of the oligonucleotide inhibitor of miR-155 is fully complementary to a mature sequence of human miR-155-5p. In various embodiments, the mature sequence of human miR-155-5p to which the sequence of the oligonucleotide inhibitor of miR-155 for use in the methods of the present invention is partially, substantially, or fully complementary to includes nucleotides 1-17, or nucleotides 2-17, or nucleotides 2-16, or nucleotides 2-15, or nucleotides 2-14, or nucleotides 2-13, or nucleotides 2-12 from the 5′ end of SEQ ID NO: 1. In one embodiment, the mature sequence of human miR-155-5p to which the sequence of the oligonucleotide inhibitor of miR-155 for use in the methods of the present invention is partially, substantially, or fully complementary to includes nucleotides 2-15 from the 5′ end of SEQ ID NO: 1. In another embodiment, the mature sequence of human miR-155-5p to which the sequence of the oligonucleotide inhibitor of miR-155 for use in the methods of the present invention is partially, substantially, or fully complementary to includes nucleotides 2-13 from the 5′ end of SEQ ID NO: 1.

In one embodiment, the oligonucleotide inhibitor of miR-155 contains at least one backbone modification, such as at least one phosphorothioate, morpholino, or phosphonocarboxylate internucleotide linkage (see, for example, U.S. Pat. Nos. 6,693,187 and 7,067,641, which are herein incorporated by reference in their entireties). In some embodiments, the phosphorothioate internucleotide linkage is a chiral phosphorothioate internucleotide linkage (see, for example, International Publication Nos. WO/2015/107425 and WO/2015/108048, which are herein incorporated by reference in their entireties). Exemplary chiral phosphorothioate intemucleotide linkages are shown as follows:

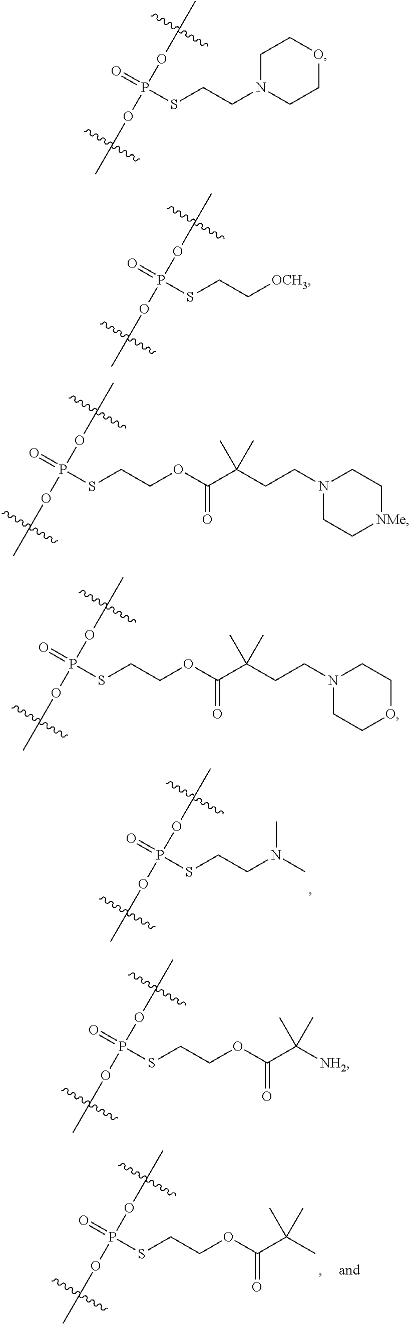

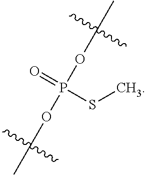 [SMILES: *OP(=O)(O*)SC]

In certain embodiments, the oligonucleotide inhibitor of miR-155 is fully phosphorothioate-linked. In some embodiments, one or more of the phosphorothioate intemucleotide linkages is a chiral phosphorothioate internucleotide linkage. In some embodiments, chirality of the phosphorothioate internucleotide linkages affects one or more properties of the oligonucleotide inhibitor of miR-155 selected from efficacy, lipophilicity, binding affinity, and stability.

In one embodiment, the oligonucleotide inhibitor of miR-155 contains at least one modified nucleotide. In some embodiments, the oligonucleotide inhibitor contains at least 5, 6, 7, 8, 9, 10, or more modified nucleotides. The term “modified nucleotide” as used herein encompasses nucleotides with sugar, base, and/or backbone modifications. Examples of modified nucleotides include, but are not limited to, locked nucleotides (LNA), ethylene-bridged nucleotides (ENA), 2′-C-bridged bicyclic nucleotide (CBBN), 2′, 4′-constrained ethyl nucleic acid called S-cEt or cEt, 2′-4′-carbocyclic LNA, and 2′ substituted nucleotides.

The terms “locked nucleotide,” “locked nucleic acid unit,” “locked nucleic acid residue,” or “LNA unit” may be used interchangeably throughout the disclosure and refer to a bicyclic nucleoside analogue. For instance, suitable oligonucleotide inhibitors can be comprised of one or more “conformationally constrained” or bicyclic sugar nucleoside modifications (BSN) that confer enhanced thermal stability to complexes formed between the oligonucleotide containing BSN and their complementary target strand. In one embodiment, the oligonucleotide inhibitors contain locked nucleotides or LNAs containing the 2′-O, 4′-C-methylene ribonucleoside (structure A) wherein the ribose sugar moiety is in a “locked” conformation. In another embodiment, the oligonucleotide inhibitors contain at least one 2′-C, 4′-C-bridged 2′ deoxyribonucleoside (structure B). See, e.g., U.S. Pat. No. 6,403,566 and Wang et al. (1999) Bioorganic and Medicinal Chemistry Letters, Vol. 9: 1147-1150, both of which are herein incorporated by reference in their entireties. In yet another embodiment, the oligonucleotide inhibitors contain at least one modified nucleoside having the structure shown in structure C. The oligonucleotide inhibitors targeting miR-155 can contain combinations of BSN (LNA, 2′-C, 4′-C-bridged 2′ deoxyribonucleoside, and the like) or other modified nucleotides, and ribonucleotides or deoxyribonucleotides.

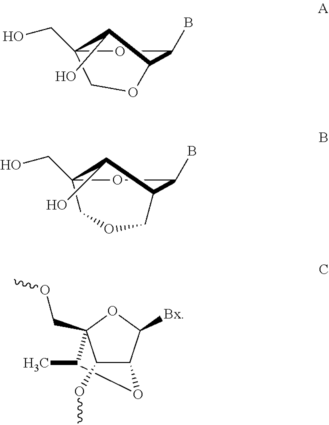

The terms “non-LNA nucleotide”, and “non-LNA modification” as used herein refer to a nucleotide different from a LNA nucleotide, i.e. the terms include a DNA nucleotide, an RNA nucleotide as well as a modified nucleotide where a base and/or sugar is modified except that the modification is not a LNA modification.

In some embodiments, the oligonucleotide inhibitor of miR-155 contains at least one nucleotide containing a non-LNA modification. For example, in one embodiment, the oligonucleotide inhibitor of miR-155 contains at least one 2′-C-bridged bicyclic nucleotide (CBBN) as described in U.S. Pre-Grant Publication No. 2016/0010090A1 (“the '090 publication”), which is hereby incorporated by reference herein in its entirety. The '090 publication describes a variety of CBBN modifications such as 2′-CBBN, oxoCBBN, amino CBBN, thioCBBN, etc. All CBBN modifications described in the '090 publications could be used in the oligonucleotide inhibitors of the present invention. In another embodiment, the non-LNA modification present in the oligonucleotide inhibitor of miR-155 could be an ethylene-bridged nucleic acid (ENA) modification. For example, in one embodiment, the oligonucleotide inhibitor of miR-155 contains at least one ethylene-bridged nucleic acid (ENA), also referred to herein as ethylene-bridged nucleotide. Other bridged modifications include 2′, 4′-constrained ethyl nucleic acid called S-cEt or cEt and 2′-4′-carbocyclic LNA (carba-LNA).

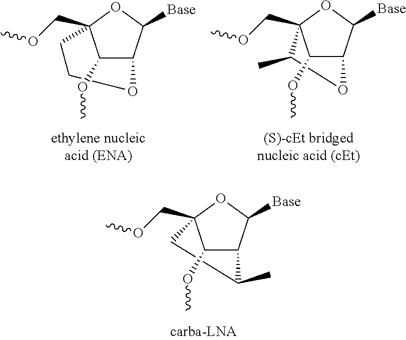

When referring to substituting a DNA or RNA nucleotide by its corresponding locked nucleotide in the context of the present invention, the term “corresponding locked nucleotide” is intended to mean that the DNA/RNA nucleotide has been replaced by a locked nucleotide containing the same naturally-occurring nitrogenous base as the DNA/RNA nucleotide that it has replaced or the same nitrogenous base that is chemically modified. For example, the corresponding locked nucleotide of a DNA nucleotide containing the nitrogenous base C may contain the same nitrogenous base C or the same nitrogenous base C that is chemically modified, such as 5-methylcytosine.

In certain embodiments, the oligonucleotide inhibitor of miR-155 contains at least 5, 6, 7, 8, 9, 10, or 11 locked nucleotides. In one embodiment, the oligonucleotide inhibitor of miR-155 contains at least 7, 8, 9, or 10 locked nucleotides. In one embodiment, at least the first three nucleotides from the 3′ end of the oligonucleotide inhibitor are locked nucleotides. In another embodiment, at least the first four nucleotides from the 3′ end of the oligonucleotide inhibitor are locked nucleotides. In yet another embodiment, the first nucleotide from the 5′ end of the oligonucleotide inhibitor is a locked nucleotide.

In certain embodiments, the oligonucleotide inhibitor contains at least 1, at least 2, at least 3, at least 4, or at least 5 DNA nucleotides. In one embodiment, at least the second nucleotide from the 5′ end of the oligonucleotide inhibitor is a DNA nucleotide. In another embodiment, at least the second and fourth nucleotides from the 5′ end of the oligonucleotide inhibitor are DNA nucleotides.

Oligonucleotide inhibitors for use in the methods of the present invention may include modified nucleotides that have a base modification or substitution. The natural or unmodified bases in RNA are the purine bases adenine (A) and guanine (G), and the pyrimidine bases cytosine (C) and uracil (U) (DNA has thymine (T)). Modified bases, also referred to as heterocyclic base moieties, include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. In certain embodiments, oligonucleotide inhibitors targeting miR-155 comprise one or more BSN modifications in combination with a base modification (e.g. 5-methylcytosine).

Oligonucleotide inhibitors for use in the methods of the present invention may include nucleotides with modified sugar moieties. Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an oligonucleotide inhibitor may have one or more nucleotides with a sugar modification at one position and also one or more nucleotides with a sugar modification at a different position.

Sugar modifications contemplated in the oligonucleotide inhibitors for use in the methods of the present invention include, but are not limited to, a substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted with C₁ to C₁₀ alkyl or C₂ to C₁₀ alkenyl and alkynyl. In one embodiment, the modification includes 2′-methoxyethoxy (2′-O—CH₂CH₂OCH₃, which is also known as 2′-O-(2-methoxyethyl) or 2′-MOE), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Additional sugar substituent groups include allyl (—CH₂—CH═CH₂), —O-allyl, methoxy (—O—CH₃), aminopropoxy (—OCH₂CH₂CH₂NH₂), and fluoro (F). Sugar substituent groups on the 2′ position (2′-) may be in the arabino (up) position or ribo (down) position. One 2′-arabino modification is 2′-F. Other similar modifications may also be made at other positions on the sugar moiety, particularly the 3′ position of the sugar on the 3′ terminal nucleoside or in 2′-5′ linked oligonucleotides and the 5′ position of 5′ terminal nucleotide. In certain embodiments, the sugar modification is a 2′-O-alkyl (e.g. 2′-O-methyl, 2′-O-methoxyethyl), 2′-halo (e.g., 2′-fluoro, 2′-chloro, 2′-bromo), and 4′ thio modifications.

Other modifications of oligonucleotide inhibitors to enhance stability and improve efficacy, such as those described in U.S. Pat. No. 6,838,283, which is herein incorporated by reference in its entirety, are known in the art and are suitable for use in the methods of the invention. For instance, to facilitate in vivo delivery and stability, the oligonucleotide inhibitor can be linked to a steroid, such as cholesterol moiety, a vitamin, a fatty acid, a carbohydrate or glycoside, a peptide, or other small molecule ligand at its 3′ end.

In some embodiments, the oligonucleotide inhibitors for use in the methods of the present invention may be conjugated to a carrier molecule such as a steroid (cholesterol). The carrier molecule is attached to the 3′ or 5′ end of the oligonucleotide inhibitor either directly or through a linker or a spacer group. In various embodiments, the carrier molecule is cholesterol, a cholesterol derivative, cholic acid or a cholic acid derivative. The use of carrier molecules disclosed in U.S. Pat. No. 7,202,227, which is incorporated by reference herein in its entirety, is also envisioned. In certain embodiments, the carrier molecule is cholesterol and it is attached to the 3′ or 5′ end of the oligonucleotide inhibitor through at least a six carbon linker. In some embodiments, the carrier molecule is attached to the 3′ or 5′ end of the oligonucleotide inhibitor through a six or nine carbon linker. In some embodiments, the linker is a cleavable linker. In various embodiments, the linker comprises a substantially linear hydrocarbon moiety. The hydrocarbon moiety may comprise from about 3 to about 15 carbon atoms and may be conjugated to cholesterol through a relatively non-polar group such as an ether or a thioether linkage. In certain embodiments, the hydrocarbon linker/spacer comprises an optionally substituted C₂ to C₁₅ saturated or unsaturated hydrocarbon chain (e.g. alkylene or alkenylene). A variety of linker/spacer groups described in U.S. Pre-grant Publication No. 2012/0128761, which is incorporated by reference herein in its entirety, can be used in the inhibitors utilized to carry out the methods of the present invention.

In one embodiment, the oligonucleotide inhibitor of miR-155 comprises a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of the oligonucleotide inhibitor are locked nucleotides and at least the second nucleotide from the 5′ end of the oligonucleotide inhibitor is a deoxyribonucleic acid (DNA) nucleotide. In some of these embodiments, the fourth nucleotide from the 3′ end of the oligonucleotide inhibitor is also a locked nucleotide. In some of these embodiments, at least the second and fourth nucleotides from the 5′ end of the oligonucleotide inhibitor are DNA nucleotides. In certain embodiments, the oligonucleotide inhibitor of miR-155 has a length of 12 or 14 nucleotides. In some embodiments, the oligonucleotide inhibitor contains at least 5, 6, 7, 8, 9, or 10 locked nucleotides. In further embodiments, at least the sixth and/or the eighth nucleotide from the 5′ end of the oligonucleotide inhibitor is a DNA nucleotide. In yet further embodiments, the oligonucleotide inhibitor comprises DNA nucleotides at the second, sixth, and the eighth position from the 5′ end.

In another embodiment, the oligonucleotide inhibitor of miR-155 comprises a sequence of 11 to 14 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are modified nucleotides and at least the second nucleotide from the 5′ end of the oligonucleotide inhibitor is a modified or an unmodified deoxyribonucleic acid (DNA) nucleotide.

In yet another embodiment, the oligonucleotide inhibitor of miR-155 comprises a sequence of 11 to 14 nucleotides, wherein the oligonucleotide inhibitor is fully complementary to a mature sequence of miR-155 and has a full phosphorothioate backbone; wherein at least 7 nucleotides of said oligonucleotide inhibitor are modified nucleotides and at least the second nucleotide from the 5′ end of the oligonucleotide inhibitor is a modified or an unmodified deoxyribonucleic acid (DNA) nucleotide.

In yet another embodiment, the oligonucleotide inhibitor of miR-155 comprises a sequence of 11 to 14 nucleotides, wherein the oligonucleotide inhibitor is fully complementary to a mature sequence of miR-155 and has a full phosphorothioate backbone; and wherein at least the first three nucleotides from 3′ end of said oligonucleotide inhibitor are modified nucleotides and at least the fourth and fifth nucleotides from the 5′ end of the oligonucleotide inhibitor are modified or unmodified deoxyribonucleic acid (DNA) nucleotides. In some of these embodiments, the fourth and/or the fifth DNA nucleotide from the 5′ end of the oligonucleotide inhibitor are unmodified DNA nucleotides.

In some embodiments where the oligonucleotide inhibitor is 11 to 14 nucleotides long, said inhibitor contains at least 5, 6, 7, 8, 9, or 10 modified nucleotides. In some of these embodiments, the oligonucleotide inhibitor contains 7, 8, 9, or 10 modified nucleotides. In some embodiments where the oligonucleotide inhibitor is 11 to 14 nucleotides long, at least the first three nucleotides from the 3′ end of said oligonucleotide inhibitor are modified nucleotides. In some embodiments, all modified nucleotides are locked nucleotides. In some embodiments, the 5, 6, 7, 8, 9, or 10 modified nucleotides present in the oligonucleotide inhibitors are a combination of locked nucleotides and nucleotides containing non-LNA modifications such as ethylene-bridged nucleotides, 2′-C-bridged bicyclic nucleotides, 2′-substituted nucleotides, and other sugar and/or base modifications described herein.

In some embodiments, the second nucleotide from the 5′ end of the oligonucleotide inhibitor is an unmodified deoxyribonucleic acid (DNA) nucleotide.

In one embodiment, the oligonucleotide inhibitor of miR-155 comprises a sequence of SEQ ID NO: 25. In another embodiment, the oligonucleotide inhibitor of miR-155 comprises a sequence of SEQ ID NO: 22. In yet another embodiment, the oligonucleotide inhibitor of miR-155 comprises a sequence of SEQ ID NO: 23.

In some other embodiments, the oligonucleotide inhibitor of miR-155 comprises a sequence selected from the group consisting of SEQ ID NOs: 33, 39, 43, 44, 47, 58, 84, 99, 111, 115, and 120.

In various embodiments, the oligonucleotide inhibitor of miR-155-5p has a sequence selected from Table 1.

TABlE 1

| SEQ ID NO. | Sequence (5′-3′) with modifications¹ |
|---|---|
| SEQ ID NO: 3 | 5′-lAs.dTs.dCs.dAs.lCs.lGs.dAs.lTs.dTs.lAs.lGs.dCs.lAs.dTs.lTs.lA-3′ |
| SEQ ID NO: 4 | 5′-lAs.dTs.dCs.dAs.lCs.lGs.dAs.dTs.lTs.lAs.lGs.dCs.lAs.dTs.lTs.lA-3′ |
| SEQ ID NO: 5 | 5′-lAs.lTs.dCs.dAs.dCs.lGs.dAs.lTs.dTs.lAs.lGs.dCs.lAs.dTs.lTs.lA-3′ |
| SEQ ID NO: 6 | 5′-lAs.lTs.dCs.dAs.dCs.lGs.lAs.dTs.dTs.lAs.lGs.lCs.dAs.lTs.dTs.lA-3′ |
| SEQ ID NO: 7 | 5′-lAs.dTs.dCs.dAs.lCs.lGs.dAs.lTs.dTs.lAs.lGs.dCs.lAs.lTs.dTs.lA-3′ |
| SEQ ID NO: 8 | 5′-lAs.lTs.dCs.dAs.lCs.dGs.dAs.dTs.lTs.lAs.dGs.lCs.lAs.dTs.lTs.lA-3′ |
| SEQ ID NO: 9 | 5-lAs.dTs.dCs.dAs.lCs.dGs.lAs.dTs.lTs.lAs.dGs.lCs.lAs.dTs.lTs.lA-3 |
| SEQ ID NO: 10 | 5′-lAs.dTs.dCs.lAs.dCs.dGs.lAs.lTs.dTs.lAs.lGs.dCs.lAs.dTs.lTs.lA-3′ |
| SEQ ID NO: 11 | 5′-lAs.dTs.lCs.dAs.dCs.lGs.dAs.lTs.lTs.dAs.dGs.lCs.lAs.dTs.lTs.lA-3′ |
| SEQ ID NO: 12 | 5′-lAs.lTs.dCs.lAs.lCs.dGs.dAs.dTs.lTs.lAs.dGs.lCs.lAs.dTs.dTs.lA-3′ |
| SEQ ID NO: 13 | 5′-lAs.dTs.lCs.dAs.dCs.dGs.lAs.dTs.lTs.lAs.dGs.lCs.lAs.dTs.lTs.lA-3′ |
| SEQ ID NO: 14 | 5′-lAs.dTs.lCs.dAs.lCs.dGs.lAs.dTs.lTs.dAs.lGs.dCs.lAs.dTs.lTs.lA-3′ |
| SEQ ID NO: 15 | 5′-lTs.dCs.dAs.lCs.dGs.dAs.lTs.dTs.dAs.lGs.dCs.lAs.lTs.dTs.lA-3′ |
| SEQ ID NO: 16 | 5′-lTs.dCs.lAs.dCs.dGs.lAs.lTs.dTs.dAs.lGs.dCs.lAs.dTs.lTs.lA-3′ |
| SEQ ID NO: 17 | 5′-lTs.dCs.dAs.dCs.lGs.lAs.lTs.dTs.dAs.lGs.dCs.lAs.dTs.lTs.lA-3′ |
| SEQ ID NO: 18 | 5′-lTs.lCs.lAs.dCs.lGs.dAs.dTs.lTs.lAs.dGs.lCs.dAs.dTs.lTs.lA-3′ |
| SEQ ID NO: 19 | 5′-lTs.dCs.dAs.lCs.dGs.dAs.dTs.lTs.lAs.lGs.lCs.lAs.lTs.lTs.lA-3′ |
| SEQ ID NO: 20 | 5′-lTs.dCs.lAs.dCs.lGs.lAs.lTs.dTs.dAs.lGs.lCs.lAs.dTs.lTs.lA-3′ |
| SEQ ID NO: 21 | 5′-lGs.lAs.lTs.lTs.lAs.lGs.dCs.lAs.lTs.dTs.lA-3′ |
| SEQ ID NO: 22 | 5′-lCs.dGs.lAs.lTs.lTs.lAs.lGs.dCs.lAs.lTs.lTs.lA-3′ |
| SEQ ID NO: 23 | 5′-lCs.dGs.lAs.lTs.lTs.dAs.lGs.dCs.lAs.lTs.lTs.lA-3′ |
| SEQ ID NO: 24 | 5′-lCs.lAs.dCs.lGs.dAs.lTs.lTs.dAs.lGs.dCs.lAs.lTs.lTs.lA-3′ |
| SEQ ID NO: 25 | 5′-lCs.dAs.lCs.dGs.dAs.lTs.lTs.dAs.lGs.dCs.lAs.lTs.lTs.lA-3′ |
| SEQ ID NO: 26 | 5′-lTs.dCs.lAs.mdCs.lGs.lAs.lTs.dTs.dAs.lGs.lCs.lAs.dTs.lTs.lA-3′ |
| SEQ ID NO: 27 | 5′-lTs.lAs.lGs.lCs.lAs.lTs.lTs.lA-3′ |
| SEQ ID NO: 29 | 5′-lCs.dAs.lCs.dGs.lAs.lTs.lTs.dAs.lGs.dCs.lAs.lTs.lTs.lA-3′ |
| SEQ ID NO: 30 | 5′-lCs.dAs.lCs.dGs.lAs.dTs.lTs.dAs.lGs.dCs.lAs.lTs.lTs.lA-3′ |
| SEQ ID NO: 31 | 5′-lCs.dAs.lCs.dGs.dAs.lTs.lTs.lAs.lGs.dCs.lAs.dTs.lTs.lA-3′ |
| SEQ ID NO: 32 | 5′-dCs.dAs.lCs.dGs.dAs.lTs.lTs.dAs.lGs.dCs.lAs.lTs.lTs.lA-3′ |
| SEQ ID NO: 33 | 5′-lCs.lAs.lCs.dGs.dAs.lTs.lTs.dAs.lGs.dCs.lAs.lTs.lTs.lA-3′ |
| SEQ ID NO: 34 | 5′-lCs.dAs.dCs.dGs.dAs.lTs.lTs.dAs.lGs.dCs.lAs.lTs.lTs.lA-3′ |
| SEQ ID NO: 35 | 5′-lCs.dAs.lCs.lGs.dAs.lTs.lTs.dAs.lGs.dCs.lAs.lTs.lTs.lA-3′ |
| SEQ ID NO: 36 | 5′-lCs.dAs.lCs.dGs.lAs.lTs.lTs.dAs.lGs.dCs.lAs.lTs.lTs.lA-3′ |
| SEQ ID NO: 37 | 5′-lCs.dAs.lCs.dGs.dAs.dTs.lTs.dAs.lGs.dCs.lAs.lTs.lTs.lA-3′ |
| SEQ ID NO: 38 | 5′-lCs.dAs.lCs.dGs.dAs.lTs.dTs.dAs.lGs.dCs.lAs.lTs.lTs.lA-3′ |
| SEQ ID NO: 39 | 5′-lCs.dAs.lCs.dGs.dAs.lTs.lTs.lAs.lGs.dCs.lAs.lTs.lTs.lA-3′ |
| SEQ ID NO: 40 | 5′-lCs.dAs.lCs.dGs.dAs.lTs.lTs.dAs.dGs.dCs.lAs.lTs.lTs.lA-3′ |
| SEQ ID NO: 41 | 5′-lCs.dAs.lCs.dGs.dAs.lTs.lTs.dAs.lGs.lCs.lAs.lTs.lTs.lA-3′ |
| SEQ ID NO: 42 | 5′-lCs.dAs.lCs.dGs.dAs.lTs.lTs.dAs.lGs.dCs.dAs.lTs.lTs.lA-3′ |
| SEQ ID NO: 43 | 5′-lCs.dAs.lCs.dGs.dAs.lTs.lTs.dAs.lGs.dCs.lAs.dTs.lTs.lA-3′ |
| SEQ ID NO: 44 | 5′-lCs.dAs.lCs.dGs.dAs.lTs.lTs.dAs.lGs.dCs.lAs.lTs.dTs.lA-3′ |
| SEQ ID NO: 45 | 5′-lCs.dAs.lCs.dGs.dAs.lTs.lTs.dAs.lGs.dCs.lAs.lTs.lTs.dA-3′ |
| SEQ ID NO: 46 | 5′-dCs.lAs.lCs.dGs.dAs.lTs.lTs.dAs.lGs.dCs.lAs.lTs.lTs.lA-3′ |
| SEQ ID NO: 47 | 5′-lCs.lAs.dCs.dGs.dAs.lTs.lTs.dAs.lGs.dCs.lAs.lTs.lTs.lA-3′ |
| SEQ ID NO: 48 | 5′-lCs.dAs.dCs.lGs.dAs.lTs.lTs.dAs.lGs.dCs.lAs.lTs.lTs.lA-3′ |
| SEQ ID NO: 49 | 5′-lCs.dAs.lCs.dGs.lAs.dTs.lTs.dAs.lGs.dCs.lAs.lTs.lTs.lA-3′ |
| SEQ ID NO: 50 | 5′-lCs.dAs.lCs.dGs.dAs.lTs.dTs.lAs.lGs.dCs.lAs.lTs.lTs.lA-3′ |
| SEQ ID NO: 51 | 5′-lCs.dAs.lCs.dGs.dAs.lTs.lTs.lAs.dGs.dCs.lAs.lTs.lTs.lA-3′ |
| SEQ ID NO: 52 | 5′-lCs.dAs.lCs.dGs.dAs.lTs.lTs.dAs.dGs.lCs.lAs.lTs.lTs.lA-3′ |
| SEQ ID NO: 53 | 5′-lCs.dAs.lCs.dGs.dAs.lTs.lTs.dAs.lGs.lCs.dAs.lTs.lTs.lA-3′ |
| SEQ ID NO: 54 | 5′-lAs.lCs.dGs.dAs.lTs.lTs.dAs.lGs.dCs.lAs.lTs.lTs.lA-3′ |
| SEQ ID NO: 55 | 5′-lCs.dGs.dAs.lTs.lTs.dAs.lGs.dCs.lAs.lTs.lTs.lA-3′ |
| SEQ ID NO: 56 | 5′-lGs.dAs.lTs.lTs.dAs.lGs.dCs.lAs.lTs.lTs.lA-3′ |
| SEQ ID NO: 57 | 5′-dAs.lCs.dGs.dAs.lTs.lTs.dAs.lGs.dCs.lAs.lTs.lTs.lA-3′ |
| SEQ ID NO: 58 | 5′-lAs.dCs.dGs.dAs.lTs.lTs.dAs.lGs.dCs.lAs.lTs.lTs.lA-3′ |
| SEQ ID NO: 59 | 5′-lAs.lCs.lGs.dAs.lTs.lTs.dAs.lGs.dCs.lAs.lTs.lTs.lA-3′ |
| SEQ ID NO: 60 | 5′-lAs.lCs.dGs.lAs.lTs.lTs.dAs.lGs.dCs.lAs.lTs.lTs.lA-3′ |
| SEQ ID NO: 61 | 5′-lAs.lCs.dGs.dAs.dTs.lTs.dAs.lGs.dCs.lAs.lTs.lTs.lA-3′ |
| SEQ ID NO: 62 | 5′-lAs.lCs.dGs.dAs.lTs.dTs.dAs.lGs.dCs.lAs.lTs.lTs.lA-3′ |
| SEQ ID NO: 63 | 5′-lAs.lCs.dGs.dAs.lTs.lTs.lAs.lGs.dCs.lAs.lTs.lTs.lA-3′ |
| SEQ ID NO: 64 | 5′-lAs.lCs.dGs.dAs.lTs.lTs.dAs.dGs.dCs.lAs.lTs.lTs.lA-3′ |
| SEQ ID NO: 65 | 5′-lAs.lCs.dGs.dAs.lTs.lTs.dAs.lGs.lCs.lAs.lTs.lTs.lA-3′ |
| SEQ ID NO: 66 | 5′-lAs.lCs.dGs.dAs.lTs.lTs.dAs.lGs.dCs.dAs.lTs.lTs.lA-3′ |
| SEQ ID NO: 67 | 5′-lAs.lCs.dGs.dAs.lTs.lTs.dAs.lGs.dCs.lAs.dTs.lTs.lA-3′ |
| SEQ ID NO: 68 | 5′-lAs.lCs.dGs.dAs.lTs.lTs.dAs.lGs.dCs.lAs.lTs.dTs.lA-3′ |
| SEQ ID NO: 69 | 5′-lAs.lCs.dGs.dAs.lTs.lTs.dAs.lGs.dCs.lAs.lTs.lTs.dA-3′ |
| SEQ ID NO: 70 | 5′-lAs.dCs.lGs.dAs.lTs.lTs.dAs.lGs.dCs.lAs.lTs.lTs.lA-3′ |
| SEQ ID NO: 71 | 5′-lAs.lCs.dGs.lAs.dTs.lTs.dAs.lGs.dCs.lAs.lTs.lTs.lA-3′ |
| SEQ ID NO: 72 | 5′-lAs.lCs.dGs.dAs.lTs.lTs.dAs.lGs.dCs.lAs.lTs.lTs.lA-3′ |
| SEQ ID NO: 73 | 5′-lAs.lCs.dGs.dAs.lTs.dTs.lAs.lGs.dCs.lAs.lTs.lTs.lA-3′ |
| SEQ ID NO: 74 | 5′-lAs.lCs.dGs.dAs.lTs.lTs.lAs.dGs.dCs.lAs.lTs.lTs.lA-3′ |
| SEQ ID NO: 75 | 5′-lAs.lCs.dGs.dAs.lTs.lTs.dAs.dGs.lCs.lAs.lTs.lTs.lA-3′ |
| SEQ ID NO: 76 | 5′-lAs.lCs.dGs.dAs.lTs.lTs.dAs.lGs.lCs.dAs.lTs.lTs.lA-3′ |
| SEQ ID NO: 77 | 5′-dCs.dGs.dAs.lTs.lTs.dAs.lGs.dCs.lAs.lTs.lTs.lA-3′ |
| SEQ ID NO: 78 | 5′-lCs.lGs.dAs.lTs.lTs.dAs.lGs.dCs.lAs.lTs.lTs.lA-3′ |
| SEQ ID NO: 79 | 5′-lCs.dGs.lAs.lTs.lTs.dAs.lGs.dCs.lAs.lTs.lTs.lA-3′ |
| SEQ ID NO: 80 | 5′-lCs.dGs.dAs.dTs.lTs.dAs.lGs.dCs.lAs.lTs.lTs.lA-3′ |
| SEQ ID NO: 81 | 5′-lCs.dGs.dAs.lTs.dTs.dAs.lGs.dCs.lAs.lTs.lTs.lA-3′ |
| SEQ ID NO: 82 | 5′-lCs.dGs.dAs.lTs.lTs.lAs.lGs.dCs.lAs.lTs.lTs.lA-3′ |
| SEQ ID NO: 83 | 5′-lCs.dGs.dAs.lTs.lTs.dAs.dGs.dCs.lAs.lTs.lTs.lA-3′ |
| SEQ ID NO: 84 | 5′-lCs.dGs.dAs.lTs.lTs.dAs.lGs.lCs.lAs.lTs.lTs.lA-3′ |
| SEQ ID NO: 85 | 5′-lCs.dGs.dAs.lTs.lTs.dAs.lGs.dCs.dAs.lTs.lTs.lA-3′ |
| SEQ ID NO: 86 | 5′-lCs.dGs.dAs.lTs.lTs.dAs.lGs.dCs.lAs.dTs.lTs.lA-3′ |
| SEQ ID NO: 87 | 5′-lCs.dGs.dAs.lTs.lTs.dAs.lGs.dCs.lAs.lTs.dTs.lA-3′ |
| SEQ ID NO: 88 | 5′-lCs.dGs.dAs.lTs.lTs.dAs.lGs.dCs.lAs.lTs.lTs.dA-3′ |
| SEQ ID NO: 89 | 5′-dCs.lGs.dAs.lTs.lTs.dAs.lGs.dCs.lAs.lTs.lTs.lA-3′ |
| SEQ ID NO: 90 | 5′-lCs.dGs.lAs.dTs.lTs.dAs.lGs.dCs.lAs.lTs.lTs.lA-3′ |
| SEQ ID NO: 91 | 5′-lCs.dGs.dAs.lTs.dTs.lAs.lGs.dCs.lAs.lTs.lTs.lA-3′ |
| SEQ ID NO: 92 | 5′-lCs.dGs.dAs.lTs.lTs.lAs.dGs.dCs.lAs.lTs.lTs.lA-3′ |
| SEQ ID NO: 93 | 5′-lCs.dGs.dAs.lTs.lTs.dAs.dGs.lCs.lAs.lTs.lTs.lA-3′ |
| SEQ ID NO: 94 | 5′-lCs.dGs.dAs.lTs.lTs.dAs.lGs.lCs.dAs.lTs.lTs.lA-3′ |
| SEQ ID NO: 95 | 5′-dGs.dAs.lTs.lTs.dAs.lGs.dCs.lAs.lTs.lTs.lA-3′ |
| SEQ ID NO: 96 | 5′-lGs.lAs.lTs.lTs.dAs.lGs.dCs.lAs.lTs.lTs.lA-3′ |
| SEQ ID NO: 97 | 5′-lGs.dAs.dTs.lTs.dAs.lGs.dCs.lAs.lTs.lTs.lA-3′ |
| SEQ ID NO: 98 | 5′-lGs.dAs.lTs.dTs.dAs.lGs.dCs.lAs.lTs.lTs.lA-3′ |
| SEQ ID NO: 99 | 5′-lGs.dAs.lTs.lTs.lAs.lGs.dCs.lAs.lTs.lTs.lA-3′ |
| SEQ ID NO: | 5′-lGs.dAs.lTs.lTs.dAs.dGs.dCs.lAs.lTs.lTs.lA-3′ |
| 100 | |
| SEQ ID NO: | 5′-lGs.dAs.lTs.lTs.dAs.lGs.lCs.lAs.lTs.lTs.lA-3′ |
| 101 | |
| SEQ ID NO: | 5′-lGs.dAs.lTs.lTs.dAs.lGs.dCs.dAs.lTs.lTs.lA-3′ |
| 102 | |
| SEQ ID NO: | 5′-lGs.dAs.lTs.lTs.dAs.lGs.dCs.lAs.dTs.lTs.lA-3′ |
| 103 | |
| SEQ ID NO: | 5′-lGs.dAs.lTs.lTs.dAs.lGs.dCs.lAs.lTs.dTs.lA-3′ |
| 104 | |
| SEQ ID NO: | 5′-lGs.dAs.lTs.lTs.dAs.lGs.dCs.lAs.lTs.lTs.dA-3′ |
| 105 | |
| SEQ ID NO: | 5′-dGs.lAs.lTs.lTs.dAs.lGs.dCs.lAs.lTs.lTs.lA-3′ |
| 106 | |
| SEQ ID NO: | 5′-lGs.lAs.dTs.lTs.dAs.lGs.dCs.lAs.lTs.lTs.lA-3′ |
| 107 | |
| SEQ ID NO: | 5′-lGs.dAs.lTs.dTs.lAs.lGs.dCs.lAs.lTs.lTs.lA-3′ |
| 108 | |
| SEQ ID NO: | 5′-lGs.dAs.lTs.lTs.lAs.dGs.dCs.lAs.lTs.lTs.lA-3′ |
| 109 | |
| SEQ ID NO: | 5′-lGs.dAs.lTs.lTs.dAs.dGs.lCs.lAs.lTs.lTs.lA-3′ |
| 110 | |
| SEQ ID NO: | 5′-lGs.dAs.lTs.lTs.dAs.lGs.lCs.dAs.lTs.lTs.lA-3′ |
| 111 | |
| SEQ ID NO: | 5′-eCs.dAs.eCs.dGs.dAs.eTs.eTs.dAs.eGs.dCs.eAs.eTs.eTs.eA-3′ |
| 112 | |
| SEQ ID NO: | 5′-lCs.dAs.lCs.dGs.dAs.lTs.lTs.dAs.lGs.dCs.eAs.lTs.lTs.eA-3′ |
| 113 | |
| SEQ ID NO: | 5′-eCs.dAs.eCs.dGs.dAs.lTs.lTs.dAs.lGs.dCs.lAs.lTs.lTs.lA-3′ |
| 114 | |
| SEQ ID NO: | 5′-lCs.dAs.lCs.dGs.dAs.lTs.lTs.dAs.eGs.dCs.lAs.lTs.lTs.lA-3′ |
| 115 | |
| SEQ ID NO: | 5′-lCs.dAs.lCs.dGs.dAs.eTs.eTs.dAs.lGs.dCs.lAs.eTs.eTs.lA-3′ |
| 116 | |
| SEQ ID NO: | 5′-lCs.dAs.lCs.dGs.dAs.lTs.eTs.dAs.lGs.dCs.lAs.lTs.lTs.lA-3′ |
| 117 | |
| SEQ ID NO: | 5′-lCs.dAs.lCs.dGs.dAs.lTs.lTs.dAs.lGs.dCs.lAs.lTs.eTs.lA-3′ |
| 118 | |
| SEQ ID NO: | 5′-lCs.dAs.lCs.dGs.dAs.lTs.lTs.dAs.lGs.dCs.abAs.lTs.lTs.abA-3′ |
| 119 | |
| SEQ ID NO: | 5′-abCs.dAs.abCs.dGs.dAs.abTs.abTs.dAs.abGs.dCs.abAs.abTs.abTs.abA- |
| 120 | 3′ |

¹1 = locked nucleic acid modification;
d = deoxyribonucleotide;
s = phosphorothioate linkage;
md = 5-Methylcytosine;
e = ethylene-bridged nucleotide (ENA)
ab = amino-2′-C-Bridged Bicyclic Nucleotide (CBBN).

In some embodiments, certain oligonucleotide inhibitors used in the methods of the present invention may show a greater inhibition of the activity or function of miR-155 in cancer cells, such as malignant T cells, e.g., CTCL cells, compared to other miR-155 inhibitors. The term “other miR-155 inhibitors” includes nucleic acid inhibitors such as antisense oligonucleotides, antimiRs, antagomiRs, mixmers, gapmers, aptamers, ribozymes, small interfering RNAs, or small hairpin RNAs; antibodies or antigen binding fragments thereof; and/or drugs, which inhibit the activity or function of miR-155. It is possible that a particular oligonucleotide inhibitor may show a greater inhibition of miR-155 in cancer cells, such as malignant T cells, compared to other oligonucleotide inhibitors. The term “greater” as used herein refers to quantitatively more or statistically significantly more.

In some embodiments, the methods of the present invention reduce or inhibit proliferation of cancer cells and/or induce apoptosis of cancer cells, such as malignant T cells including cutaneous T cell lymphoma (CTCL) cells. Intralesional administration of one or more oligonucleotide inhibitors of miR-155 may provide up to about 5%, 10%, 15%, 20%, 25%, 30%, 35%, 40%, 45%, 50%, 55%, 60%, 65%, 70%, 80%, 90% or 100%, including values therebetween, reduction in the number of cancer cells. In some embodiments, intralesional administration of one or more oligonucleotide inhibitors of miR-155 may provide at least about 5%, 10%, 15%, 20%, 25%, 30%, 35%, 40%, 45%, or 50%, including values therebetween, reduction in the number of cancer cells. In one embodiment, the reduction in the number of cancer cells occurs at the site of the treated lesion. In another embodiment, the reduction in the number of cancer cells occurs at the site(s) of one or more untreated lesions. In yet another embodiment, the reduction in the number of cancer cells occurs at the site of the treated lesion as well as at the site(s) of one or more untreated lesions.

In one embodiment, the method for treating CTCL comprises administering intralesionally an oligonucleotide inhibitor of miR-155 that has a sequence of about 8 to about 22 nucleotides. In one embodiment, the oligonucleotide inhibitor of miR-155 is at least partially complementary to a mature sequence of miR-155-5p, e.g. at least about 75%, 80%, 85%, 90%, 95%, 96%, 97%, 98%, or 99% complementary to a mature sequence of miR-155-5p. In one embodiment, the oligonucleotide inhibitor comprises a sequence that is 100% or fully complementary to a mature sequence of miR-155-5p. In some embodiments, the oligonucleotide inhibitor of miR-155 comprises one or more modified nucleotides. The modified nucleotides that may be present in the oligonucleotide inhibitors of the present invention include, but are not limited to, locked nucleotides, ethylene-bridged nucleotides, 2′-C-bridged bicyclic nucleotides, 2′-substituted nucleotides, and other sugar and/or base modifications described herein.

In one embodiment, the method for treating CTCL comprises administering intralesionally an oligonucleotide inhibitor of miR-155 that has a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are locked nucleotides and at least the second nucleotide from the 5′ end of the oligonucleotide inhibitor is a deoxyribonucleic acid (DNA) nucleotide.

In another embodiment, the method for treating CTCL comprises administering intralesionally an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are modified nucleotides and at least the second nucleotide from the 5′ end of the oligonucleotide inhibitor is a deoxyribonucleic acid (DNA) nucleotide.

In yet another embodiment, the method for treating CTCL comprises administering intralesionally an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least 7 nucleotides of said oligonucleotide inhibitor are modified nucleotides and at least the second nucleotide from the 5′ end of the oligonucleotide inhibitor is a deoxyribonucleic acid (DNA) nucleotide.

In yet another embodiment, the method for treating CTCL comprises administering intralesionally an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least the first three nucleotides from 3′ end of said oligonucleotide inhibitor are modified nucleotides and at least the fourth and fifth nucleotides from the 5′ end of the oligonucleotide inhibitor are deoxyribonucleic acid (DNA) nucleotides.

In certain exemplary embodiments, the method for treating CTCL comprises administering intralesionally an oligonucleotide inhibitor of miR-155 selected from the group consisting of SEQ ID NO: 22, SEQ ID NO: 23, and SEQ ID NO: 25. In some other embodiments, the method for treating CTCL comprises administering intralesionally an oligonucleotide inhibitor of miR-155 selected from the group consisting of SEQ ID NOs: 33, 39, 43, 44, 47, 58, 84, 99, 111, 115, and 120.

In one embodiment, the invention provides methods for treating the mycosis fungoides (MF) form of CTCL by administering intralesionally an oligonucleotide inhibitor of miR-155 that has a sequence of about 8 to about 22 nucleotides. In one embodiment, the oligonucleotide inhibitor of miR-155 is at least partially complementary to a mature sequence of miR-155-5p, e.g. at least about 75%, 80%, 85%, 90%, 95%, 96%, 97%, 98%, or 99% complementary to a mature sequence of miR-155-5p. In one embodiment, the oligonucleotide inhibitor comprises a sequence that is 100% or fully complementary to a mature sequence of miR-155-5p. In some embodiments, the oligonucleotide inhibitor of miR-155 comprises one or more modified nucleotides. The modified nucleotides that may be present in the oligonucleotide inhibitors of the present invention include, but are not limited to, locked nucleotides, ethylene-bridged nucleotides, 2′-C-bridged bicyclic nucleotides, 2′-substituted nucleotides, and other sugar and/or base modifications described herein.

In one embodiment, the method for treating the MF form of CTCL comprises administering intralesionally an oligonucleotide inhibitor of miR-155 that has a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are locked nucleotides and at least the second nucleotide from the 5′ end of the oligonucleotide inhibitor is a deoxyribonucleic acid (DNA) nucleotide.

In another embodiment, the invention provides methods for treating the mycosis fungoides (MF) form of CTCL comprising administering intralesionally an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are modified nucleotides and at least the second nucleotide from the 5′ end of the oligonucleotide inhibitor is a deoxyribonucleic acid (DNA) nucleotide.

In another embodiment, the invention provides methods for treating the mycosis fungoides (MF) form of CTCL comprising administering intralesionally an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least 7 nucleotides of said oligonucleotide inhibitor are modified nucleotides and at least the second nucleotide from the 5′ end of the oligonucleotide inhibitor is a deoxyribonucleic acid (DNA) nucleotide.

In yet another embodiment, the invention provides methods for treating the mycosis fungoides (MF) form of CTCL comprising administering intralesionally an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least the first three nucleotides from 3′ end of said oligonucleotide inhibitor are modified nucleotides and at least the fourth and fifth nucleotides from the 5′ end of the oligonucleotide inhibitor are deoxyribonucleic acid (DNA) nucleotides.

In certain embodiments, the method for treating the MF form of CTCL comprises administering intralesionally an oligonucleotide inhibitor of miR-155 selected from the group consisting of SEQ ID NO: 22, SEQ ID NO: 23, and SEQ ID NO: 25. In some other embodiments, the method for treating the MF form of CTCL comprises administering intralesionally an oligonucleotide inhibitor of miR-155 selected from the group consisting of SEQ ID NOs: 33, 39, 43, 44, 47, 58, 84, 99, 111, 115, and 120.

The invention also encompasses methods for treating CTCL comprising intralesionally administering an oligonucleotide inhibitor of miR-155 in combination with the administration of a second therapeutic agent. In an exemplary embodiment, the second therapeutic agent is administered subcutaneously or intravenously. Current treatments for CTCL include skin-directed therapies such as topical steroids, topical nitrogen mustard (mechlorethamine HCL), topical retinoids, phototherapy, ultraviolet light treatment, psoralen ultraviolet light treatment, radiotherapy, electron beam therapy, etc. and systemic therapies such as administration of histone deacetylase (HDAC) inhibitors, retinoids (bexarotene), interferon, and low dose antifolates (e.g. methotrexate and pralatrexate). Additional treatment options such as anti-CD30 antibody (e.g. Brentuximab), anti-CCR4 antibody (e.g. mogamulizumab), and anti-PD-1 or anti-PD-L1 antibody are currently being tested. The second therapeutic agent generally comprises an agent or a therapy selected from one of these treatments. For example, the invention encompasses methods for treating CTCL by administering the oligonucleotide inhibitor of miR-155 in combination with a second therapy such as treatment with HDAC inhibitors, retinoids, interferon, antifolates, topical steroids, topical retinoids, topical nitrogen mustard, phototherapy, ultraviolet light, psoralen and ultraviolet light, radiotherapy, electron beam therapy, anti-CD30 antibody (e.g. Brentuximab), anti-CCR4 antibody (e.g. mogamulizumab), and anti-PD-1 or anti-PD-L1 antibody.

In some embodiments, the second therapeutic agent is an oligonucleotide inhibitor of miR-155. In one embodiment, the oligonucleotide inhibitor of miR-155 that is administered intralesionally is the same oligonucleotide inhibitor of miR-155 that is administered subcutaneously and/or intravenously. In an alternative embodiment, the oligonucleotide inhibitor of miR-155 that is administered intralesionally is different than the oligonucleotide inhibitor of miR-155 that is administered subcutaneously and/or intravenously.

In other embodiments, the second therapeutic agent is a retinoid or a histone deacetylase (HDAC) inhibitor. A variety of HDAC inhibitors are known, some of which are approved by FDA for clinical use and some are being tested in clinical trials. The methods for treating cancer according to the invention encompass the use of HDAC inhibitors including, but not limited to, vorinostat, romidepsin, panobinostat (LBH589), mocetinostat, belinostat (PXD101), abexinostat, CI-994 (tacedinaline), and MS-275 (entinostat). In embodiments where a second therapy/agent is included, the second therapy/agent may be administered at different times prior to or after administration of the oligonucleotide inhibitor of miR-155. Prior administration includes, for instance, administration of the first agent within the range of about one week to up to 30 minutes prior to administration of the second agent. Prior administration may also include, for instance, administration of the first agent within the range of about 2 weeks to up to 30 minutes prior to administration of the second agent. After or later administration includes, for instance, administration of the second agent within the range of about one week to up to 30 minutes after administration of the first agent. After or later administration may also include, for instance, administration of the second agent within the range of about 2 weeks to up to 30 minutes after administration of the first agent.

The invention also provides methods for reducing or inhibiting proliferation of cancer cells, particularly malignant T cells (e.g., CTCL cells), by intralesionally administering an oligonucleotide inhibitor of miR-155 that has a sequence of about 8 to about 22 nucleotides. In one embodiment, the oligonucleotide inhibitor of miR-155 is at least partially complementary to a mature sequence of miR-155-5p, e.g. at least about 75%, 80%, 85%, 90%, 95%, 96%, 97%, 98%, or 99% complementary to a mature sequence of miR-155-5p. In one embodiment, the oligonucleotide inhibitor comprises a sequence that is 100% or fully complementary to a mature sequence of miR-155-5p. In some embodiments, the oligonucleotide inhibitor of miR-155 comprises one or more modified nucleotides. The modified nucleotides that may be present in the oligonucleotide inhibitors of the present invention include, but are not limited to, locked nucleotides, ethylene-bridged nucleotides, 2′-C-bridged bicyclic nucleotides, 2′-substituted nucleotides, and other sugar and/or base modifications described herein

In one embodiment, the invention provides methods for reducing or inhibiting proliferation of cancer cells, particularly malignant T cells (e.g., CTCL cells), by intralesionally administering an oligonucleotide inhibitor of miR-155 that has a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are locked nucleotides and at least the second nucleotide from the 5′ end of the oligonucleotide inhibitor is a deoxyribonucleic acid (DNA) nucleotide.

In another embodiment, the invention provides methods for reducing or inhibiting proliferation of cancer cells, particularly malignant T cells (e.g., CTCL cells), by intralesionally administering an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are modified nucleotides and at least the second nucleotide from the 5′ end of the oligonucleotide inhibitor is a deoxyribonucleic acid (DNA) nucleotide.

In another embodiment, the invention provides methods for reducing or inhibiting proliferation of cancer cells, particularly malignant T cells (e.g., CTCL cells), by intralesionally administering an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least 7 nucleotides of said oligonucleotide inhibitor are modified nucleotides and at least the second nucleotide from the 5′ end of the oligonucleotide inhibitor is a deoxyribonucleic acid (DNA) nucleotide.

In yet another embodiment, the invention provides methods for reducing or inhibiting proliferation of cancer cells, particularly malignant T cells (e.g., CTCL cells), by intralesionally administering an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least the first three nucleotides from 3′ end of said oligonucleotide inhibitor are modified nucleotides and at least the fourth and fifth nucleotides from the 5′ end of the oligonucleotide inhibitor are deoxyribonucleic acid (DNA) nucleotides.

In certain embodiments, the invention provides methods for reducing or inhibiting proliferation of cancer cells, particularly malignant T cells (e.g., CTCL cells), by intralesionally administering an oligonucleotide inhibitor of miR-155 selected from the group consisting of SEQ ID NO: 22, SEQ ID NO: 23, and SEQ ID NO: 25. In some other embodiments, the invention provides methods for reducing or inhibiting proliferation of cancer cells, particularly malignant T cells (e.g., CTCL cells), by intralesionally administering an oligonucleotide inhibitor of miR-155 selected from the group consisting of SEQ ID NOs: 33, 39, 43, 44, 47, 58, 84, 99, 111, 115, and 120.

Malignant T cells include cutaneous T cell lymphoma (CTCL) cells, CD4⁺ T cells and memory T cells. Intralesional administration of an oligonucleotide inhibitor of miR-155 reduces the activity or function of miR-155 and/or up-regulates one or more target genes of miR-155 in CTCL cells following administration. Furthermore, intralesional administration of an oligonucleotide inhibitor may blunt the inflammatory response that drives redness, itchiness, and scaling of CTCL lesions. Methods for reducing or inhibiting proliferation of CTCL cells also include the use of second therapy/agents described above along with intralesional administration of one or more oligonucleotide inhibitors of miR-155.

Preferably, intralesional administration of an oligonucleotide inhibitor of miR-155 to the subject results in the improvement of one or more symptoms or pathologies associated with CTCL. For instance, in one embodiment, intralesional administration of an oligonucleotide inhibitor miR-155 alone or in combination with the administration of a second therapeutic agent such as a HDAC inhibitor reduces the number of skin lesions; number of red, itchy patches or plaques on skin; and/or formation of new skin lesions/patches/plaques associated with CTCL. In one embodiment, intralesional administration of an oligonucleotide inhibitor of miR-155 alone or in combination with a second therapeutic agent such as a HDAC inhibitor reduces or inhibits migration of malignant T lymphocytes to the skin. In another embodiment, intralesional administration of an oligonucleotide inhibitor of miR-155 alone or in combination with a second therapeutic agent reduces total malignant T lymphocytes in the skin. In yet another embodiment, intralesional administration of an oligonucleotide inhibitor of miR-155 alone or in combination with a second therapeutic agent reduces the number of malignant T cells that may escape or migrate from the skin into the periphery.

As used herein, the term “subject” or “patient” refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In other embodiments, the subject is a human.

Any of the oligonucleotide inhibitors of miR-155 described herein can be delivered to the target cell (e.g. malignant T cells) by delivering to the cell an expression vector encoding the miR-155 oligonucleotide inhibitor. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. In one particular embodiment, the viral vector is a lentiviral vector or an adenoviral vector. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, an expression vector for expressing an oligonucleotide inhibitor of miR-155 comprises a promoter operably linked to a polynucleotide sequence encoding the oligonucleotide inhibitor. The phrase “operably linked” or “under transcriptional control” as used herein means that the promoter is in the correct location and orientation in relation to a polynucleotide to control the initiation of transcription by RNA polymerase and expression of the polynucleotide.

As used herein, a “promoter” refers to a DNA sequence recognized by the synthetic machinery of the cell, or introduced synthetic machinery, required to initiate the specific transcription of a gene. Suitable promoters include, but are not limited to RNA pol I, pol II, pol III, and viral promoters (e.g. human cytomegalovirus (CMV) immediate early gene promoter, the SV40 early promoter, and the Rous sarcoma virus long terminal repeat). In one embodiment, the promoter is a T-cell specific promoter such as the proximal and distal promoters of the lck gene or promoter and enhancer sequences of the CD4 gene, etc.

In certain embodiments, the promoter operably linked to a polynucleotide encoding a miR-155 oligonucleotide inhibitor can be an inducible promoter. Inducible promoters are known in the art and include, but are not limited to, tetracycline promoter, metallothionein IIA promoter, heat shock promoter, steroid/thyroid hormone/retinoic acid response elements, the adenovirus late promoter, and the inducible mouse mammary tumor virus LTR.

Methods of delivering expression constructs and nucleic acids to cells are known in the art and can include, for example, calcium phosphate co-precipitation, electroporation, microinjection, DEAE-dextran, lipofection, transfection employing polyamine transfection reagents, cell sonication, gene bombardment using high velocity microprojectiles, and receptor-mediated transfection.

In some embodiments, the oligonucleotide inhibitor of miR-155 is formulated with a pharmaceutically acceptable carrier or excipient. Accordingly, the invention also provides a method of treating cutaneous T-cell lymphoma (CTCL) in a subject in need thereof, wherein the method comprises intralesionally administering to the subject a pharmaceutical composition comprising an oligonucleotide inhibitor of miR-155 as disclosed herein and a pharmaceutically acceptable carrier or excipient.

In one embodiment, pharmaceutical composition comprises an effective dose of the oligonucleotide inhibitor of miR-155, wherein the oligonucleotide inhibitor of miR-155 comprises a sequence of about 8 nucleotides to about 22 nucleotides that is at least partially complementary to a mature sequence of miR-155-5p, e.g. at least about 75%, 80%, 85%, 90%, 95%, 96%, 97%, 98%, or 99% complementary to a mature sequence of miR-155-5p. In one embodiment, the oligonucleotide inhibitor comprises a sequence that is 100% or fully complementary to a mature sequence of miR-155-5p. In some embodiments, the oligonucleotide inhibitor of miR-155 comprises one or more modified nucleotides.

In another embodiment, the pharmaceutical composition comprises an effective dose of an oligonucleotide inhibitor of miR-155 having a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of the oligonucleotide inhibitor are locked nucleotides and at least the second nucleotide from the 5′ end of the oligonucleotide inhibitor is a deoxyribonucleic acid (DNA) nucleotide.

In yet another embodiment, the pharmaceutical composition comprises an effective dose of an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are modified nucleotides and at least the second nucleotide from the 5′ end of the oligonucleotide inhibitor is a deoxyribonucleic acid (DNA) nucleotide.

In yet another embodiment, the pharmaceutical composition comprises an effective dose of an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least 7 nucleotides of said oligonucleotide inhibitor are modified nucleotides and at least the second nucleotide from the 5′ end of the oligonucleotide inhibitor is a deoxyribonucleic acid (DNA) nucleotide.

In yet another embodiment, the pharmaceutical composition comprises an effective dose of an oligonucleotide inhibitor of miR-155 that has a sequence of 11 to 14 nucleotides, wherein the oligonucleotide inhibitor is fully complementary to a mature sequence of miR-155 and has a full phosphorothioate backbone; and wherein at least the first three nucleotides from 3′ end of said oligonucleotide inhibitor are modified nucleotides and at least the fourth and fifth nucleotides from the 5′ end of the oligonucleotide inhibitor are deoxyribonucleic acid (DNA) nucleotides.

In certain embodiments, pharmaceutical compositions comprise an effective dose of an oligonucleotide inhibitor having a sequence selected from the group consisting of SEQ ID NO: 22, SEQ ID NO: 23, and SEQ ID NO: 25. In some other embodiments, pharmaceutical compositions comprise an effective dose of an oligonucleotide inhibitor having a sequence selected from the group consisting of SEQ ID NOs: 33, 39, 43, 44, 47, 58, 84, 99, 111, 115, and 120. In yet other embodiments, the pharmaceutical composition comprises an oligonucleotide inhibitor having a sequence selected from the sequences listed in Table 1.

An “effective dose” is an amount sufficient to effect a beneficial or desired clinical result. An effective dose of an oligonucleotide inhibitor of miR-155 may be from about 1 mg/kg to about 100 mg/kg, about 2.5 mg/kg to about 50 mg/kg, or about 5 mg/kg to about 25 mg/kg. In some embodiments, an effective dose may be about 18.75, 37.5, or 75 mg of the oligonucleotide inhibitor per skin lesion of the patient. The precise determination of what would be considered an effective dose may be based on factors individual to each patient, including their size, age, type of disorder, and form of inhibitor (e.g. naked oligonucleotide or an expression construct etc.). Therefore, dosages can be readily ascertained by those of ordinary skill in the art from this disclosure and the knowledge in the art.

For intralesional administration, liquid injectable pharmaceutically acceptable compositions are generally used. Such compositions can, for example, be prepared by diluting the oligonucleotide inhibitor with sterile preservative free water or saline to produce an isotonic solution containing the appropriate concentration of inhibitor. Other injectable compositions using aqueous dextrose, glycerol, ethanol and the like, to thereby form a solution or suspension for injection can also be used. If desired, minor amounts of nontoxic auxiliary substances such as wetting or emulsifying agents, preservatives, pH buffering agents and the like, for example, sodium acetate or sorbitan monolaurate, can be incorporated into the compositions. Actual methods of preparing such dosage forms are known, or will be apparent, to those skilled in this art; see for example, Remington's Pharmaceutical Sciences, Mack Publishing Company, Easton, P.A., 15th Edition, 1975. The amount of administered is critical only to the extent that it is effective for the therapeutic purpose. The quantity in the composition or formulation administered will, in any event, be an amount effective to achieve an effect against CTCL in the subject being treated.

In one embodiment, the oligonucleotide inhibitor of miR-155 is formulated for administration at a concentration of about 10 mg/mL to a concentration of about 500 mg/mL. In another embodiment, the oligonucleotide inhibitor of miR-155 is formulated for administration at a concentration of about 20 mg/mL to a concentration of about 200 mg/mL. In an exemplary embodiment, the oligonucleotide inhibitor of miR-155 is formulated for administration at a concentration of about 75 mg/mL. In another exemplary embodiment, the oligonucleotide inhibitor of miR-155 is formulated for administration at a concentration of about 150 mg/mL.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids), or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, compositions are preferably administered in a manner compatible with the dosage formulation and in such amount as is therapeutically effective. The formulations may easily be administered in a variety of dosage forms such as injectable solutions, cream, ointment, paste, lotion, or gel and the like. For parenteral administration in an aqueous solution, for example, the solution generally is suitably buffered and the liquid diluent first rendered isotonic for example with sufficient saline or glucose. Such aqueous solutions may be used, for example, for intravenous, subcutaneous, and intradermal administration. Preferably, sterile aqueous media are employed as is known to those of skill in the art, particularly in light of the present disclosure. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by regulatory agencies.

In certain embodiments of the invention, the pharmaceutical compositions of the invention are packaged with or stored within a device for administration. Devices for injectable formulations include, but are not limited to, pre-filled syringes, injection ports, autoinjectors, injection pumps, and injection pens. Devices for aerosolized or powder formulations include, but are not limited to, inhalers, insufflators, aspirators, and the like. Devices for dermal delivery of compositions of the present invention also include dermal microneedle injection or patches. Thus, the present invention includes administration devices comprising a pharmaceutical composition of the invention for treating or preventing one or more of the disorders described herein.

In one embodiment, the invention provides topical compositions comprising the oligonucleotide inhibitors of miR-155 and one or more cosmetically or pharmaceutically acceptable carriers or excipients. The term “cosmetically acceptable” as used herein means that the carriers or excipients are suitable for use in contact with tissues (e.g., the skin) without undue toxicity, incompatibility, instability, irritation, allergic response, and the like. In some embodiments, the oligonucleotide inhibitor of miR-155 is applied directly to one or more CTCL lesions.

Cosmetic or pharmaceutical carriers or excipients can be liquids, such as water and oils, including those of petroleum, animal, vegetable or synthetic origin, such as peanut oil, soybean oil, mineral oil, sesame oil and the like. Topical compositions often comprise an oil-in-water or a water-in-oil emulsion. The invention encompasses using such emulsions for preparing topical composition of antimiR-155 compounds. Suitable pharmaceutical carriers are described in “Remington's Pharmaceutical Sciences” by E. W. Martin. Suitable cosmetic carriers are described below.

In one embodiment, the cosmetically acceptable topical carrier is from about 50% to about 99.99%, by weight, of the composition (e.g., from about 80% to about 99%, by weight, of the composition). The topical compositions include, but are not limited to, solutions, lotions, creams, gels, sticks, sprays, ointments, cleansing liquid washes, solid bars, shampoos, pastes, foams, powders, mousses, shaving creams, wipes, patches, nail lacquers, wound dressing, adhesive bandages, hydrogels, and films. These product types may comprise several types of cosmetically acceptable topical carriers including, but not limited to solutions, emulsions (e.g., microemulsions and nanoemulsions), gels, solids and liposomes. Certain non-limitative examples of such carriers are set forth hereinbelow. Other suitable carriers may be formulated by those of ordinary skill in the art.

Topical compositions useful in the present invention may be formulated as a solution comprising an emollient. Such compositions preferably contain from about 1% to about 50% of an emollient(s). As used herein, the term “emollient” refers to materials used for the prevention or relief of dryness, as well as for the protection of the skin. A number of suitable emollients are known and may be used in the present invention. For example, Sagarin, Cosmetics, Science and Technology, 2nd Edition, Vol. 1, pp. 32-43 (1972) and the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (hereinafter “ICI Handbook”) contains numerous examples of suitable materials.

A lotion can be made from such a solution. Lotions typically comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may comprise a simple base of animal or vegetable oils or semi-solid hydrocarbons. An ointment may comprise from about 2% to about 10% of an emollient(s) plus from about 0.1% to about 2% of a thickening agent(s). A more complete disclosure of thickening agents or viscosity increasing agents useful herein can be found in Sagarin, Cosmetics, Science and Technology, 2nd Edition, Vol. 1, pp. 72-73 (1972) and the ICI Handbook pp. 1693-1697.

The topical compositions useful in the present invention may be formulated as emulsions. If the carrier is an emulsion, from about 1% to about 10% (e.g., from about 2% to about 5%) of the carrier comprises an emulsifier(s). Emulsifiers may be nonionic, anionic or cationic. Suitable emulsifiers are disclosed in, for example, in McCutcheon's Detergents and Emulsifiers, North American Edition, pp. 317-324 (1986), and the ICI Handbook, pp. 1673-1686.

Lotions and creams can be formulated as emulsions. Typically such lotions comprise from 0.5% to about 5% of an emulsifier(s). Such creams would typically comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well known in the cosmetic art and are useful in the present invention. Multiphase emulsion compositions, for example the water-in-oil-in-water type, as disclosed in U.S. Pat. Nos. 4,254,105 and 4,960,764, may also be useful in the present invention. In general, such single or multiphase emulsions contain water, emollients, and emulsifiers as essential ingredients.

The topical compositions of this invention can also be formulated as a gel (e.g., an aqueous, alcohol, alcohol/water, or oil gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents.

Liposomal formulations are also useful compositions of the subject invention. In one embodiment, the oligonucleotides are contained within the liposome. Examples of liposomes are unilamellar, multilamellar, and paucilamellar liposomes, which may or may not contain phospholipids. Such compositions can be prepared by combining the oligonucleotide inhibitor with a phospholipid, such as dipalmitoylphosphatidyl choline, cholesterol and water. Commercially available fat emulsions that may be suitable for delivering the nucleic acids of the invention to cancer cells or the skin tissue include Intralipid®, Liposyn®, Liposyn® II, Liposyn® III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. Nos. 5,981,505; 6,217,900; 6,383,512; 5,783,565; 7,202,227; 6,379,965; 6,127,170; 5,837,533; 6,747,014; and WO03/093449, which are herein incorporated by reference in their entireties.

The liposome preparation may then be incorporated into one of the above carriers (e.g., a gel or an oil-in-water emulsion) in order to produce the liposomal formulation. Other compositions and uses of topically applied liposomes are described in Mezei, M., “Liposomes as a Skin Drug Delivery System”, Topics in Pharmaceutical Sciences (D. Breimer and P. Speiser, eds.), Elsevier Science Publishers B. V., New York, N.Y., 1985, pp. 345-358, PCT Patent Application No. WO96/31194, Niemiec, et al., 12 Pharm. Res. 1184-88 (1995), and U.S. Pat. No. 5,260,065.

In one embodiment, the liposomes are present in the topical composition in an amount, based upon the total volume of the composition, of from about 5 mg/ml to about 100 mg/ml such as from about 10 mg/ml to about 50 mg/ml.

In addition to the above carriers and excipients, other emollients and surface active agents can be incorporated in the emulsions, including glycerol trioleate, acetylated sucrose distearate, sorbitan trioleate, polyoxyethylene (1) monostearate, glycerol monooleate, sucrose distearate, polyethylene glycol (50) monostearate, octylphenoxypoly (ethyleneoxy) ethanol, decaglycerin penta-isostearate, sorbitan sesquioleate, hydroxylated lanolin, lanolin, triglyceryl diisostearate, polyoxyethylene (2) oleyl ether, calcium stearoyl-2-lactylate, methyl glucoside sesquistearate, sorbitan monopalmitate, methoxy polyethylene glycol-22/dodecyl glycol copolymer (Elfacos E200), polyethylene glycol-45/dodecyl glycol copolymer (Elfacos ST9), polyethylene glycol 400 distearate, and lanolin derived sterol extracts, glycol stearate and glycerol stearate; alcohols, such as cetyl alcohol and lanolin alcohol; myristates, such as isopropyl myristate; cetyl palmitate; cholesterol; stearic acid; propylene glycol; glycerine, sorbitol and the like.

In certain embodiments, liposomes used for delivery are amphoteric liposomes such SMARTICLES® (Marina Biotech, Inc.) which are described in detail in U.S. Pre-grant Publication No. 20110076322. The surface charge on the SMARTICLES® is fully reversible which make them particularly suitable for the delivery of nucleic acids. SMARTICLES® can be delivered via injection, remain stable, and aggregate free and cross cell membranes to deliver the nucleic acids.

One will generally desire to employ appropriate salts and buffers to render delivery vehicles stable and allow for uptake by target cells. Pharmaceutical compositions of the present invention comprise an effective amount of the delivery vehicle comprising the inhibitor polynucleotides (e.g. liposomes or other complexes or expression vectors) dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or polynucleotides of the compositions.

The present application provides kits comprising one or more miR-155 inhibitors described herein. In some embodiments, the kits further contain a pharmaceutically acceptable excipient and instruction manual. In one specific embodiment, the kit comprises any one or more of the miR-155 inhibitor compositions described herein, with one or more pharmaceutically acceptable excipients. The present application also provides articles of manufacture comprising any one of the therapeutic compositions or kits described herein. Examples of an article of manufacture include vials (including sealed vials).

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

### EXAMPLES

### Example 1: Intra-Tumoral Injection of a miR-155 Inhibitor Results in Regression of the Injected Lesion as Well as Non-Injected Lesions

### Methodology:

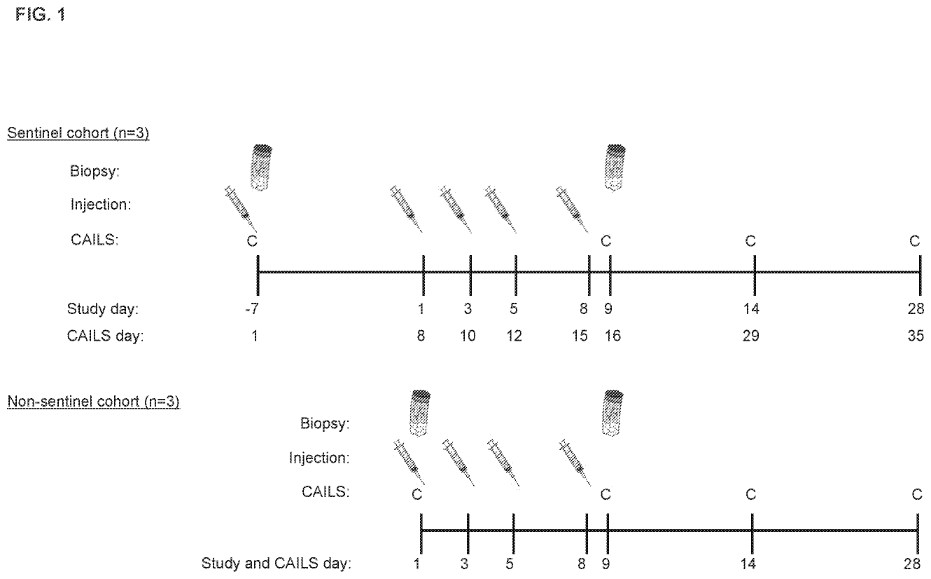

FIG. 1 provides the study design of the clinical trial. The clinical trial described below employed a dose-escalation to evaluate both intratumoral and subcutaneous administration of a miR-155 inhibitor at doses of 75 mg and up to 900 mg per injection, respectively. Patients were required to be ≥18 years old, have a confirmed diagnosis of MF, be clinical stage I-III with plaques or tumors, be on a stable treatment regimen or without any concomitant therapy for MF, and have no other major illness.

6 patients were dosed with four or five 75 mg intratumoral injections of a miR-155 inhibitor (SEQ ID NO: 25) over 2 weeks. In addition, 4 patients received saline injections in a second lesion on the same schedule. Skin biopsies were taken from miR-155 inhibitor and saline treated lesions for molecular, bioanalytical, and histological analyses, before the first dose and after the last dose.

### Results:

Six patients (5M/1F, median age 61 years, 5 Caucasian/1 African-American) were dosed intratumorally. All tolerated the administrations well with only minimal injection site reactions noted in three patients. One patient was discontinued from the trial due to rapid progression of disease, which was considered not related to the study drug. There were no clinically significant adverse events or laboratory abnormalities. Table 2 provides the demographic and clinical characteristics of patients who received the miR-155 inhibitor.

TABLE 2

| Characteristic | miR-155 Inhibitor-treated (n = 6) |
|---|---|
| Age, years | |
| Median (Range) | 61 (50-64) |
| Sex | |
| Male | 5 |
| Female | 1 |
| Race | |
| White | 4 |
| African American- | 1 |
| Hispanic | 1 |
| Stage at screening | |
| IB | 1 |
| IIA | 2 |
| IIB | 3 |
| Prior systemic treatments | |
| Median (range) | 3 (1-6) |
| Reported concomitant therapies | |
| Topical | 1 (0-2) |
| Systemic | 1 (0-1) |

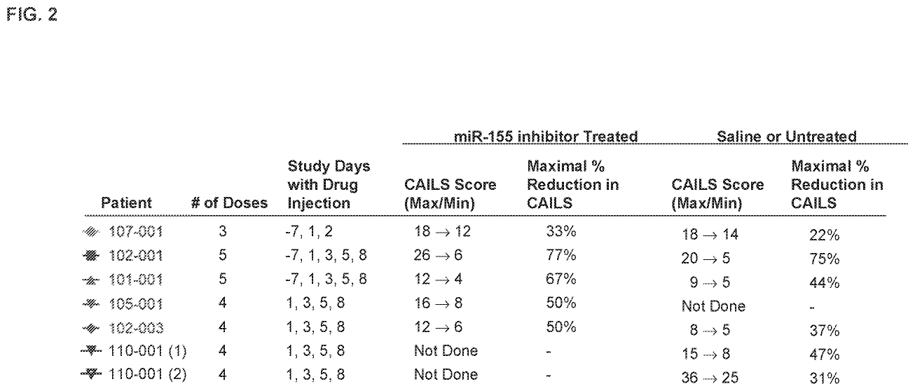

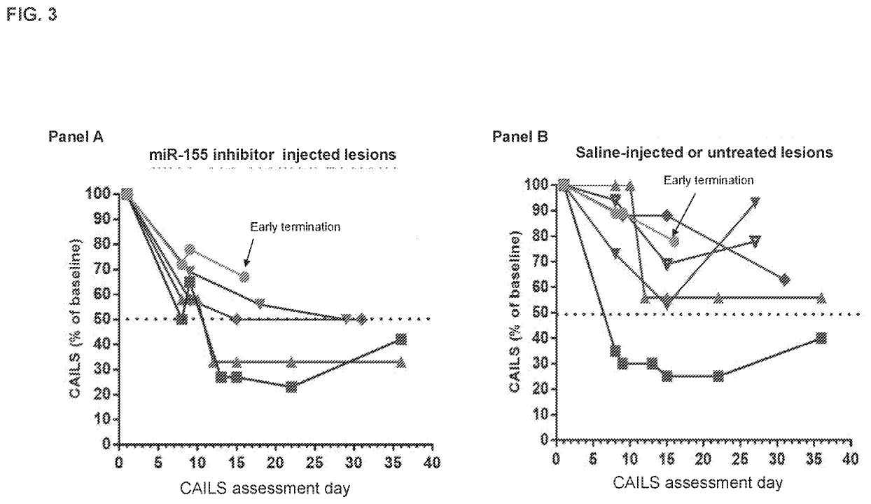

FIG. 2 and FIG. 3 FIG. 3 show the efficacy of intratumoral injection of the miR-155 inhibitor.

All patients showed a reduction in the baseline Composite Assessment of Index Lesion Severity (CAILS) score in both miR-155 inhibitor-treated and saline-treated lesions.

The individual lesion CAILS score was obtained by adding the severity score of each of the following categories: erythema, scaling, plaque elevation, and surface area. The maximum score achievable is 50. The maximum (baseline) CAILS score and the minimum score recorded for each monitored lesion, as well as the calculated maximum percentage change, is shown. The change over time in CAILS scores (normalized to 100% at baseline) is presented graphically. Panel A of FIG. 3 shows the scores for miR-155 inhibitor-injected lesions and Panel B of FIG. 3 shows the score for the saline-injected or un-injected lesions.

All lesions showed improvement in the CAILS score. A reduction in CAILS score of ≥50% (consistent with the definition of clinical response (Olsen et al., 2011)) was observed in the miR-155 inhibitor treated lesions in all four evaluable subjects who completed dosing. In contrast, a ≥50% reduction was observed in only one saline treated lesion. The miR-155 inhibitor injected lesion responses were maintained to the End of Study visit (either 28 days or 35 days after the first dose).

The maximal reduction was on average 55% [range: 33% to 77%] in the miR-155 inhibitor treated lesion and 43% [range: 22% to 75%] in the saline treated lesions. In all the subjects that completed dosing, the miR-155 inhibitor treated lesions had a CAILS score reduction of ≥50% which was maintained to the end of study; in contrast, a ≥50% reduction was observed in only one saline treated lesion.

Most patients noted a marked decrease in systemic pruritus. Histological examination of pre-treatment and post-treatment biopsies of the same lesion injected with miR-155 inhibitor from five evaluable patients revealed that one patient had a complete loss of the neoplastic infiltrate, two patients had a reduction in neoplastic cell infiltrate density and depth, one patient had fewer CD30+ large atypical cells, and one patient demonstrated no change.

Notably, lesions found the patient's chest, abdomen, and arm showed reduced redness, height, and borders (i.e., surface area). These results show that intra-tumoral administration of a miR-155 inhibitor can produce a broad clinical response on multiple lesions.

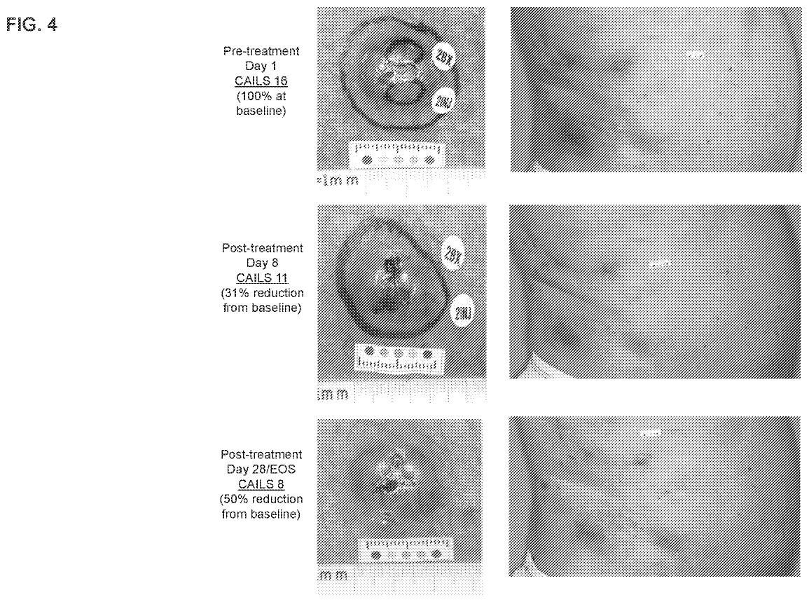

FIG. 4 shows a photographic example of a clinical response for a patient in the clinical trial. A 50-year old male patient (ID 105-001) with stage IIB mycosis fungoides showed improvement in skin lesions after four intratumoral injections of the miR-155 inhibitor.

After the first dose, miR-155 inhibitor had a median t_{1/2} in plasma of 4.8 hours and a mean Cₘₐₓ of 1.2 μg/mL. The drug was detectable 24 hours after the last dose in the miR-155 inhibitor-injected lesions that were biopsied. Table 3 shows the pharmacokinetic characteristics of the intratumoral injection of the miR-155 inhibitor.

TABLE 3

| | | Tₘₐₓ | Cₘₐₓ | t_{1/2} | AUC_{inf} | Cl/F |
|---|---|---|---|---|---|---|
| Cohort | Patient # | (h) | (μg/mL) | (h) | (μg*hr/mL) | (L/h) |
| Sentinel | 101-001 | 1 | 1.15 | 4.84 | 11.2 | 6.71 |
| | 102-001 | 1 | 0.721 | 4.12 | 5.88 | 12.8 |
| | 107-001 | 0.5 | 2.28 | 4.29 | 7.63 | 9.83 |
| | Mean | N/A* | 1.38 | 4.41 | 8.23 | 9.77 |
| | SD | N/A | 0.805 | 0.378 | 2.7 | 3.02 |
| Non-sentinel | 102-003 | 0.5 | 1.95 | 5.42 | 7.79 | 9.63 |
| | 105-001 | 1 | 0.562 | UND† | UND | UND |
| | 110-001 | 0.5 | 0.782 | 10.7 | 5.08 | 14.8 |
| | Mean | N/A | 1.1 | 8.06 | 6.44 | 12.2 |
| | SD | N/A | 0.746 | 3.73 | 1.91 | 3.62 |

*Not calculated for categorical variables
†UND = Undetermined due to undefined terminal elimination rate constant

Gene expression analysis of the pre- and post-treatment biopsies showed transcript changes consistent with the expected mechanism of action of miR-155 inhibitor.

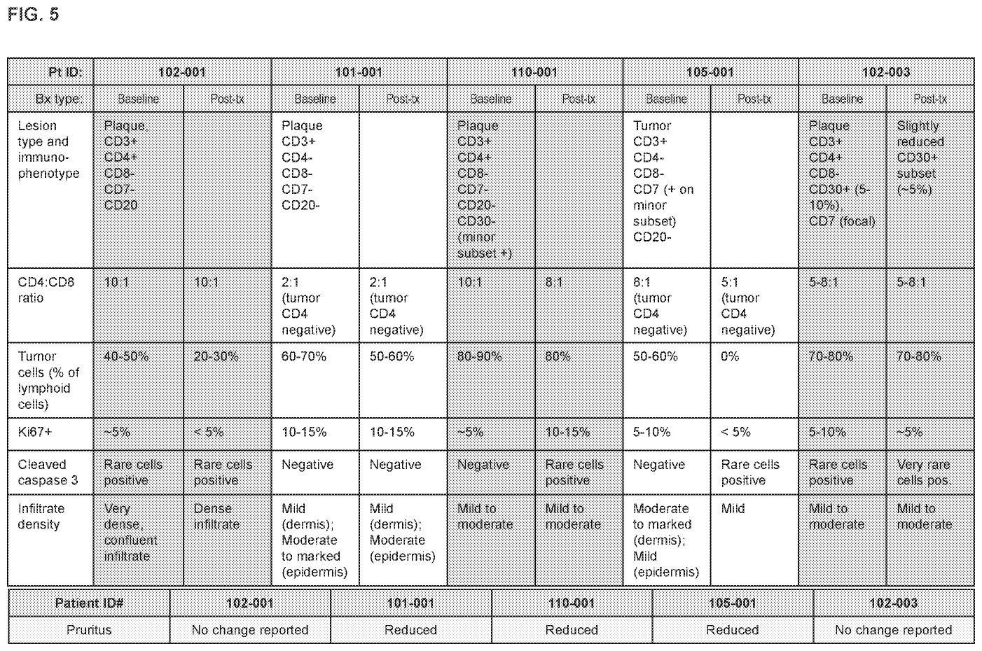

FIG. 5 shows the histological findings and changes in pruritus after 8 or 15 days of miR-155 treatment. Baseline and post-treatment biopsies of the miR-155 inhibitor-injected lesion were taken from 5 of 6 subjects. H&E and immunohistochemical staining for CD4, CD8, CD7, CD3, CD20, Ki67, and cleaved caspase 3 was performed followed by interpretation by a blinded hematopathologist. Improvements in pruritus were reported for three of the five patients who completed dosing.

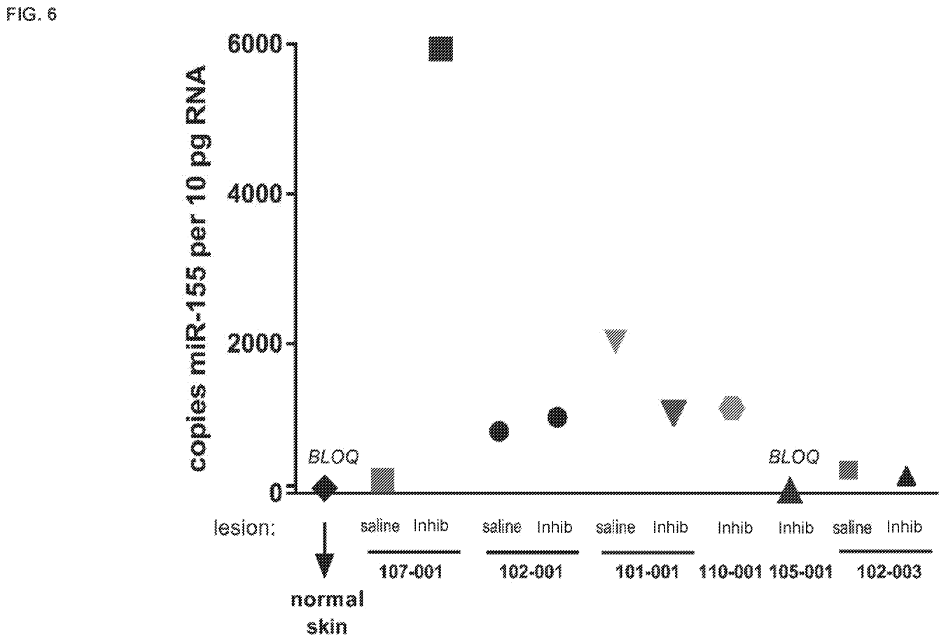

FIG. 6 shows miR-155 copy number in baseline lesion biopsies. miR-155 was quantitated by qPCR calibrated against a standard curve. The levels of miR-155 in baseline biopsies varied from below limit of quantitation (patient 105-001) to 5936 copies/10 pg RNA (patient 107-001). miR-155 in normal skin is typically below the limit of quantitation of the assay. In the x-axis of the figure, “Saline” indicates saline-treated subjects; “Inhib” indicates miR-155 inhibitor treated subjects.

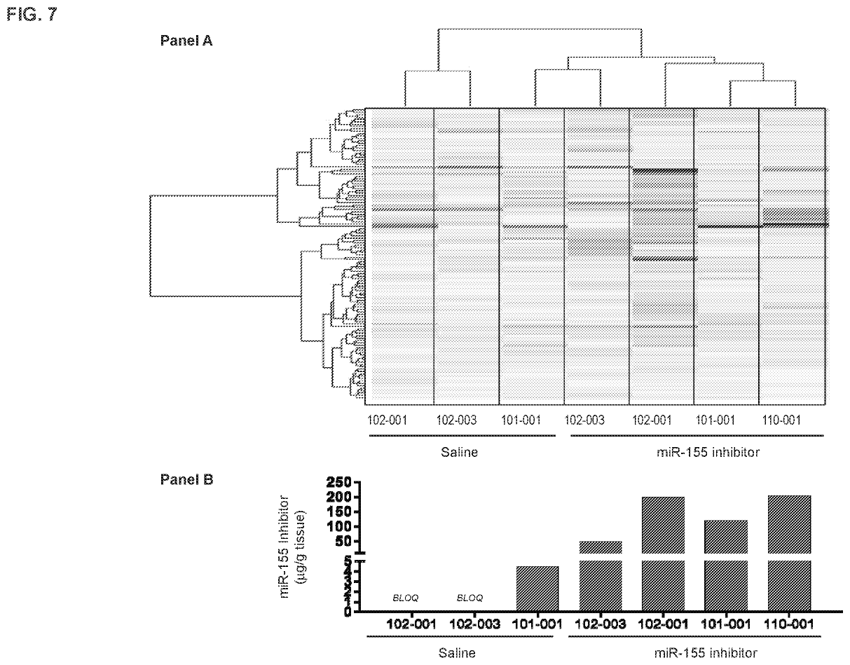

FIG. 7 shows the gene expression changes common to mycosis fungoides lesion biopsies with intratumoral injection of the miR-155 inhibitor. miR-155 inhibitor treatment of three mycosis fungoides cell lines previously identified 587 transcripts with changed expression levels compared to untreated cells (Seto et al. 2015). 122 of these 587 genes were found to be consistently up-regulated or consistently down-regulated in 4 of the 5 patients' biopsies collected after treatment with the miR-155 inhibitor. FIG. 7, Panel A shows the heat map of the fold-change of 122 genes in the miR-155 inhibitor treated lesion biopsies normalized to the pre-treatment biopsy taken from the same lesion. Two-way hierarchical clustering was performed on the signatures, revealing two clusters of samples: The first cluster represents the gene expression of lesions injected with saline, whereas the second cluster represents the expression in all lesions injected with the miR-155 inhibitor and one saline-injected lesion. FIG. 7, Panel B shows a bar graph of the miR-155 inhibitor quantification in the biopsies, as mg of miR-155 inhibitor per gram of tissue. One saline-treated biopsy demonstrated a gene signature that showed some similarity to the miR-155 inhibitor common signature, consistent with the detectable amount of miR-155 inhibitor in the tissue (101-001, first bar). This shows distal distribution of the drug from the miR-155 inhibitor-treated lesion.

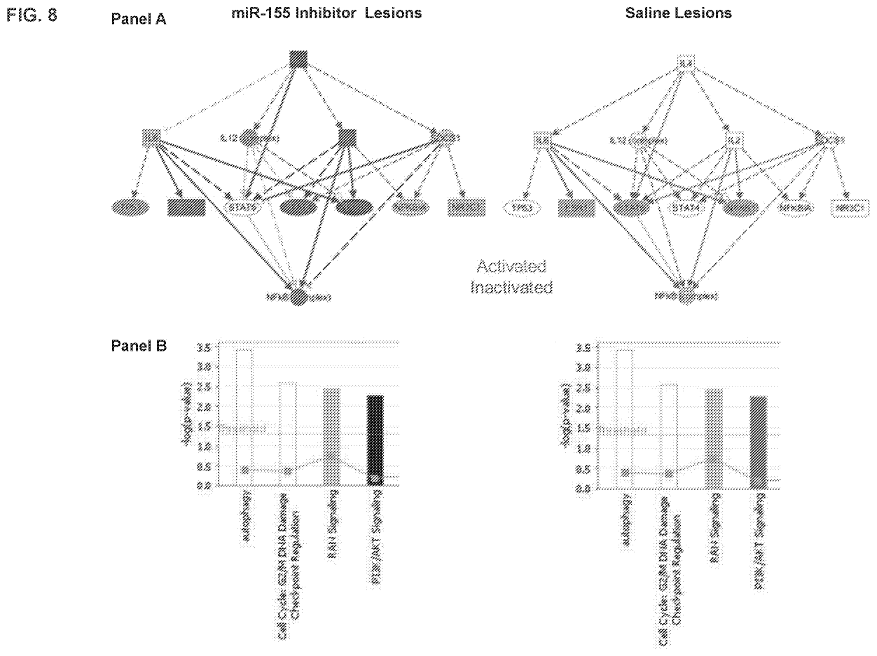

FIG. 8 shows that miR-155 inhibitor treatment inactivates the STAT, NFkB, and PI3K/AKT pathways. Ingenuity Pathway Analysis (IPA) identified the biological context of the gene expression analysis. IPA analysis of the common signature that was changed with miR-155 inhibitor treatment in four biopsies were associated with inactivation of the STAT, NFκB, and PI3K/AKT pathways, consistent with the mechanism of miR-155 inhibition (Panel A, left). In contrast, these pathways are activated in the saline-treated lesions (Panel A, right). IPA's Canonical Pathway Analysis was utilized to identify pathways enriched in the common set of genes regulated by miR-155 inhibitor treatment. Shown in Panel B are the top four pathways enriched in the common gene signature, according to p-value (the higher number on the bar graph indicates greater enrichment; the threshold line indicates the p-value threshold above which enrichment is considered significant). Of these four pathways, the PI3K/AKT pathway is predicted to be inhibited following miR-155 inhibitor treatment, based on the differential regulation of the genes associated with this pathway. In contrast, the PI3K/AKT pathway is predicted to be activated in saline-treated lesions. Additionally, the gene signature in the miR-155 inhibitor treated lesions reflected increased cell death.

### CONCLUSIONS

Intratumoral injection of the miR-155 inhibitor of SEQ ID NO: 25 was well-tolerated, and demonstrated therapeutic improvements in cutaneous lesions, based on CAILS scores and histological findings. In addition, reductions in CAILS scores in other lesions as well as decreases in systemic symptoms such as pruritus were observed. Preliminary biomarker analysis shows that the miR-155 inhibitor of SEQ ID NO: 25 induces transcriptional changes consistent with on-target activity.

While the described invention has been described with reference to the specific embodiments thereof it should be understood by those skilled in the art that various changes may be made and equivalents may be substituted without departing from the true spirit and scope of the invention. The various embodiments described above can be combined to provide further embodiments. In addition, many modifications may be made to adopt a particular situation, material, composition of matter, process, process step or steps, to the objective spirit and scope of the described invention. All such modifications are intended to be within the scope of the claims appended hereto.

1. A method of treating cutaneous T-cell lymphoma (CTCL) in a subject in need thereof, wherein the method comprises administering to the subject an oligonucleotide inhibitor of miR-155, wherein the inhibitor is administered intralesionally.

2. The method of claim 1, wherein the inhibitor is administered via intralesional injection.

3. The method of claim 1, wherein the CTCL is the mycosis fungoides (MF) form of CTCL.

4. The method of claim 1, wherein the intralesional administration reduces the redness, thickness, height, scaling, and/or surface area of one or more untreated lesions on the skin of said subject.

5. The method of claim 1, wherein the oligonucleotide inhibitor of miR-155 is formulated for administration at a concentration of about 10 mg/mL to a concentration of about 500 mg/mL.

6. The method of claim 5, wherein the oligonucleotide inhibitor of miR-155 is formulated for administration at a concentration of about 20 mg/mL to a concentration of about 200 mg/mL.

7. The method of claim 6, wherein the oligonucleotide inhibitor of miR-155 is formulated for administration at a concentration of about 75 mg/mL.

8. The method of claim 6, wherein the oligonucleotide inhibitor of miR-155 is formulated for administration at a concentration of about 150 mg/mL.

9. The method of claim 1, wherein the oligonucleotide inhibitor of miR-155 is formulated with a pharmaceutically acceptable carrier or excipient.

10-18. (canceled)

19. The method of claim 1, wherein said oligonucleotide inhibitor has a sequence of SEQ ID NO: 25.

20. The method of claim 1, wherein said oligonucleotide inhibitor has a sequence selected from Table 1.

21. (canceled)

22. The method of claim 1, wherein said oligonucleotide inhibitor reduces the activity or function of miR-155.

23. The method of claim 1, wherein said oligonucleotide inhibitor reduces proliferation of CTCL cells.

24. The method of claim 1, wherein said oligonucleotide inhibitor induces apoptosis of CTCL cells.

25-36. (canceled)

37. The method of claim 1, wherein said oligonucleotide inhibitor has a sequence selected from the group consisting of SEQ ID NOs: 39, 43, 44, 58, 84, 99, 111, 115, and 120.

38. (canceled)

39. The method of claim 1, further comprising administering one or more therapeutic agents subcutaneously and/or intravenously.

40. The method of claim 39, wherein said therapeutic agent is an oligonucleotide inhibitor of miR-155.

41. The method of claim 40, wherein the oligonucleotide inhibitor of miR-155 that is administered intralesionally is the same oligonucleotide inhibitor of miR-155 that is administered subcutaneously and/or intravenously.

42. The method of claim 40, wherein the oligonucleotide inhibitor of miR-155 that is administered intralesionally is different than the oligonucleotide inhibitor of miR-155 that is administered subcutaneously and/or intravenously.

43. The method of claim 39, wherein said therapeutic agent is selected from the group consisting of HDAC inhibitors, retinoids, interferon, antifolates, topical steroids, topical retinoids, topical nitrogen mustard, phototherapy, ultraviolet light, psoralen and ultraviolet light, radiotherapy, electron beam therapy, anti-CD30 antibody, anti-CCR4 antibody, anti-PD-1 antibody and anti-PD-L1 antibody.

44. The method of claim 43, wherein said therapeutic agent is a retinoid or a HDAC inhibitor.

45. The method of claim 44, wherein the retinoid is bexarotene.

46. The method of claim 44, wherein the HDAC inhibitor is selected from the group consisting of vorinostat, romidepsin, panobinostat (LBH589), mocetinostat, belinostat (PXD101), abexinostat, CI-994 (tacedinaline), and MS-275 (entinostat).

47-54. (canceled)
